SEQUENCE LISTING (94 sequences)

SEQ ID NO: 1 - protein, 76 aa, synthetic construct [sequence omitted]

SEQ ID NO: 2 - protein, 37 aa, synthetic construct
  REGION 1..37: note = Synthetic: Amino acids 1-37 of ubiquitin
MQIFVKTLTGKTITLEVEPSDTIENVKAKIQDKEGIP

SEQ ID NO: 3 - protein, 42 aa, synthetic construct
  REGION 1..42: note = Synthetic: Amino acids 35-76 of ubiquitin
GIPPDQQRLIFAGKQLEDGRTLSDYNIQKESTLHLVLRLRGG

SEQ ID NO: 4 - protein, 44 aa, synthetic construct
  REGION 1..44: note = Synthetic: sUb-HC-PGG
GIPPDQQRLIFAGKQLEDGRTLSDYNIQKESTLHLVLRLPGGGS

SEQ ID NO: 5 - protein, 44 aa, synthetic construct
  REGION 1..44: note = Synthetic: sUb-HC-GS
GIPPDQQRLIFAGKQLEDGRTLSDYNIQKESTLHLVLRLGSGGS

SEQ ID NO: 6 - protein, 49 aa, synthetic construct
  REGION 1..49: note = Synthetic: sUb-LC-IPV
MQIFVKTLTGKTITLEVEPSDTIENVKAKIQDKEGIPGGGSIPVSLRSG

SEQ ID NO: 7 - protein, 49 aa, synthetic construct
  REGION 1..49: note = Synthetic: sUb-LC-PLG
MQIFVKTLTGKTITLEVEPSDTIENVKAKIQDKEGIPGGSGPLGLAGGS

SEQ ID NO: 8 - protein, 49 aa, synthetic construct
  REGION 1..49: note = Synthetic: sUb-LC-Scr
MQIFVKTLTGKTITLEVEPSDTIENVKAKIQDKEGIPGGSGLALGPGGS

SEQ ID NO: 9 - protein, 47 aa, synthetic construct
  REGION 1..47: note = Synthetic: sUbext-LC-PGG
MQIFVKTLTGKTITLEVEPSDTIENVKAKIQDKEGIPPDQQRLGGGS

SEQ ID NO: 10 - protein, 55 aa, synthetic construct
  REGION 1..55: note = Synthetic: sUbext-LC-IPV
MQIFVKTLTGKTITLEVEPSDTIENVKAKIQDKEGIPPDQQRLGGGSIPVSLRSG

SEQ ID NO: 11 - protein, 52 aa, synthetic construct
  REGION 1..52: note = Synthetic: sUb-LC-flip
GIPPDQQRLIFAGKQLEDGRTLSDYNIQKESTLHLVLRLPGGGGIPVSLRSG

SEQ ID NO: 12 - protein, 52 aa, synthetic construct
  REGION 1..52: note = Synthetic: sUb-HC-IPV
GIPPDQQRLIFAGKQLEDGRTLSDYNIQKESTLHLVLRLGSGGSIPVSLRSG

SEQ ID NO: 13 - protein, 51 aa, synthetic construct
  REGION 1..51: note = Synthetic: sUb-HC-PLG
GIPPDQQRLIFAGKQLEDGRTLSDYNIQKESTLHLVLRLGGSGPLGLAGGS

SEQ ID NO: 14 - protein, 51 aa, synthetic construct
  REGION 1..51: note = Synthetic: sUb-HC-Scr
GIPPDQQRLIFAGKQLEDGRTLSDYNIQKESTLHLVLRLGGSGLALGPGGS

SEQ ID NO: 15 - protein, 44 aa, synthetic construct; identical to SEQ ID NO: 4
  REGION 1..44: note = Synthetic: sUbext-HC-PGG

SEQ ID NO: 16 - protein, 52 aa, synthetic construct
  REGION 1..52: note = Synthetic: sUbext-HC-IPV
GIPPDQQRLIFAGKQLEDGRTLSDYNIQKESTLHLVLRLPGGGSIPVSLRSG

SEQ ID NO: 17 - protein, 49 aa, synthetic construct
  REGION 1..49: note = Synthetic: sUb-HC-short
GIPPDQQRLIFAGKQLEDGRTLSDYNIQKESTLHLVLGGSGPLGLAGGS

SEQ ID NO: 18 - protein, 49 aa, synthetic construct
  REGION 1..49: note = Synthetic: sUb-HC-flip
MQIFVKTLTGKTITLEVEPSDTIENVKAKIQDKEGIPGGGGIPVSLRSG

SEQ ID NO: 19 - protein, 49 aa, synthetic construct
  REGION 1..49: note = Synthetic: sUb-Q2K-LC-IPV
MKIFVKTLTGKTITLEVEPSDTIENVKAKIQDKEGIPGGGSIPVSLRSG

SEQ ID NO: 20 - protein, 49 aa, synthetic construct
  REGION 1..49: note = Synthetic: sUb-Q2K-LC-PLG
MKIFVKTLTGKTITLEVEPSDTIENVKAKIQDKEGIPGGSGPLGLAGGS

SEQ ID NO: 21 - protein, 49 aa, synthetic construct
  REGION 1..49: note = Synthetic: sUb-Q2K-LC-ScR
MKIFVKTLTGKTITLEVEPSDTIENVKAKIQDKEGIPGGSGLALGPGGS

SEQ ID NO: 22 - protein, 49 aa, synthetic construct
  REGION 1..49: note = Synthetic: sUb-Q2R-LC-IPV
MRIFVKTLTGKTITLEVEPSDTIENVKAKIQDKEGIPGGGSIPVSLRSG

SEQ ID NO: 23 - protein, 49 aa, synthetic construct
  REGION 1..49: note = Synthetic: sUb-K11C-LC-IPV
MQIFVKTLTGCTITLEVEPSDTIENVKAKIQDKEGIPGGGSIPVSLRSG

SEQ ID NO: 24 - protein, 49 aa, synthetic construct
  REGION 1..49: note = Synthetic: sUb-P19C-LC-IPV
MQIFVKTLTGKTITLEVECSDTIENVKAKIQDKEGIPGGGSIPVSLRSG

SEQ ID NO: 25 - protein, 49 aa, synthetic construct
  REGION 1..49: note = Synthetic: sUb-Q31K-LC-IPV
MQIFVKTLTGKTITLEVEPSDTIENVKAKIKDKEGIPGGGSIPVSLRSG

SEQ ID NO: 26 - protein, 49 aa, synthetic construct
  REGION 1..49: note = Synthetic: sUb-Q31R-LC-IPV
MQIFVKTLTGKTITLEVEPSDTIENVKAKIRDKEGIPGGGSIPVSLRSG

SEQ ID NO: 27 - protein, 49 aa, synthetic construct
  REGION 1..49: note = Synthetic: sUb-Q31C-LC-IPV
MQIFVKTLTGKTITLEVEPSDTIENVKAKICDKEGIPGGGSIPVSLRSG

SEQ ID NO: 28 - protein, 49 aa, synthetic construct
  REGION 1..49: note = Synthetic: sUb-F4C-LC-IPV
MQICVKTLTGKTITLEVEPSDTIENVKAKIQDKEGIPGGGSIPVSLRSG

SEQ ID NO: 29 - protein, 49 aa, synthetic construct
  REGION 1..49: note = Synthetic: sUb-F4E-LC-IPV
MQIEVKTLTGKTITLEVEPSDTIENVKAKIQDKEGIPGGGSIPVSLRSG

SEQ ID NO: 30 - protein, 49 aa, synthetic construct
  REGION 1..49: note = Synthetic: sUb-F4D-LC-IPV
MQIDVKTLTGKTITLEVEPSDTIENVKAKIQDKEGIPGGGSIPVSLRSG

SEQ ID NO: 31 - protein, 49 aa, synthetic construct
  REGION 1..49: note = Synthetic: sUb-Q2K-V26F-LC-PLG
MKIFVKTLTGKTITLEVEPSDTIENFKAKIQDKEGIPGGSGPLGLAGGS

SEQ ID NO: 32 - protein, 49 aa, synthetic construct
  REGION 1..49: note = Synthetic: sUb-Q2K-V5L-V26L-LC-PLG
MKIFLKTLTGKTITLEVEPSDTIENLKAKIQDKEGIPGGSGPLGLAGGS

SEQ ID NO: 33 - protein, 54 aa, synthetic construct
  REGION 1..54: note = Synthetic: sUb-E34D-HC-IPV
GDGIPPDQQRLIFAGKQLEDGRTLSDYNIQKESTLHLVLRLGSGGSIPVSLRSG

SEQ ID NO: 34 - protein, 54 aa, synthetic construct
  REGION 1..54: note = Synthetic: sUb-E34C-HC-IPV
GCGIPPDQQRLIFAGKQLEDGRTLSDYNIQKESTLHLVLRLGSGGSIPVSLRSG

SEQ ID NO: 35 - protein, 52 aa, synthetic construct
  REGION 1..52: note = Synthetic: sUb-S57C-HC-IPV
GIPPDQQRLIFAGKQLEDGRTLCDYNIQKESTLHLVLRLGSGGSIPVSLRSG

SEQ ID NO: 36 - protein, 52 aa, synthetic construct
  REGION 1..52: note = Synthetic: sUb-P38D-HC-IPV
GIPDDQQRLIFAGKQLEDGRTLSDYNIQKESTLHLVLRLGSGGSIPVSLRSG

SEQ ID NO: 37 - protein, 52 aa, synthetic construct
  REGION 1..52: note = Synthetic: sUb-P38C-HC-IPV
GIPCDQQRLIFAGKQLEDGRTLSDYNIQKESTLHLVLRLGSGGSIPVSLRSG

SEQ ID NO: 38 - protein, 52 aa, synthetic construct
  REGION 1..52: note = Synthetic: sUb-P38E-HC-IPV
GIPEDQQRLIFAGKQLEDGRTLSDYNIQKESTLHLVLRLGSGGSIPVSLRSG

SEQ ID NO: 39 - protein, 52 aa, synthetic construct
  REGION 1..52: note = Synthetic: sUb-T66C-HC-IPV
GIPPDQQRLIFAGKQLEDGRTLSDYNIQKESCLHLVLRLGSGGSIPVSLRSG

SEQ ID NO: 40 - protein, 52 aa, synthetic construct
  REGION 1..52: note = Synthetic: sUb-T66K-HC-IPV
GIPPDQQRLIFAGKQLEDGRTLSDYNIQKESKLHLVLRLGSGGSIPVSLRSG

SEQ ID NO: 41 - protein, 52 aa, synthetic construct
  REGION 1..52: note = Synthetic: sUb-T66R-HC-IPV
GIPPDQQRLIFAGKQLEDGRTLSDYNIQKESRLHLVLRLGSGGSIPVSLRSG

SEQ ID NO: 42 - protein, 52 aa, synthetic construct
  REGION 1..52: note = Synthetic: sUb-T66K-L56K-HC-IPV
GIPPDQQRLIFAGKQLEDGRTKSDYNIQKESKLHLVLRLGSGGSIPVSLRSG

SEQ ID NO: 43 - protein, 51 aa, synthetic construct
  REGION 1..51: note = Synthetic: sUb-L43F-HC-PLG
GIPPDQQRFIFAGKQLEDGRTLSDYNIQKESTLHLVLRLGGSGPLGLAGGS

SEQ ID NO: 44 - protein, 5 aa, synthetic construct
  REGION 1..5: note = Synthetic: Linker 1
GSGGS

SEQ ID NO: 45 - protein, 4 aa, synthetic construct
  REGION 1..4: note = Synthetic: Linker 2
GGGS

SEQ ID NO: 46 - protein, 12 aa, synthetic construct
  REGION 1..12: note = Synthetic: Linker 3
SGGGGGGGGGGS

SEQ ID NO: 47 - protein, 4 aa, synthetic construct
  REGION 1..4: note = Synthetic: Linker 4
GGAA

SEQ ID NO: 48 - protein, 5 aa, synthetic construct
  REGION 1..5: note = Synthetic: Linker 5
GGGGS

SEQ ID NO: 49 - protein, 5 aa, synthetic construct
  REGION 1..5: note = Synthetic: Linker 6
LAAAA

SEQ ID NO: 50 - protein, 4 aa, synthetic construct
  REGION 1..4: note = Synthetic: Linker 7
GGSG

SEQ ID NO: 51 - protein, 5 aa, synthetic construct
  REGION 1..5: note = Synthetic: Linker 8
GGSGG

SEQ ID NO: 52 - protein, 5 aa, synthetic construct
  REGION 1..5: note = Synthetic: Linker 9
GSGSG

SEQ ID NO: 53 - protein, 5 aa, synthetic construct
  REGION 1..5: note = Synthetic: Linker 10
GSGGG

SEQ ID NO: 54 - protein, 5 aa, synthetic construct
  REGION 1..5: note = Synthetic: Linker 11
GGGSG

SEQ ID NO: 55 - protein, 5 aa, synthetic construct
  REGION 1..5: note = Synthetic: Linker 12
GSSSG

SEQ ID NO: 56 - skipped

SEQ ID NO: 57 - protein, 8 aa, synthetic construct
  REGION 1..8: note = Synthetic: IPV cleavage site
IPVSLRSG

SEQ ID NO: 58 - protein, 10 aa, synthetic construct
  REGION 1..10: note = Synthetic: Linker-IPV cleavage site
GSIPVSLRSG

SEQ ID NO: 59 - protein, 6 aa, synthetic construct
  REGION 1..6: note = Synthetic: M2 cleavage site
GPLGVR

SEQ ID NO: 60 - protein, 8 aa, synthetic construct
  REGION 1..8: note = Synthetic: MMP cleavage site 1
LSGRSDNY

SEQ ID NO: 61 - protein, 5 aa, synthetic construct
  REGION 1..5: note = Synthetic: MMP cleavage site 2
PLGVR

SEQ ID NO: 62 - protein, 6 aa, synthetic construct
  REGION 1..6: note = Synthetic: Protease site 1
PLGLAG

SEQ ID NO: 63 - protein, 6 aa, synthetic construct
  REGION 1..6: note = Synthetic: Protease-resistant site
LALGPG

SEQ ID NO: 64 - protein, 5 aa, synthetic construct
  REGION 1..5: note = Synthetic: Protease site 2
YGRAA

SEQ ID NO: 65 - protein, 4 aa, synthetic construct
  REGION 1..4: note = Synthetic: Stub sequence
LRSG

SEQ ID NO: 66 - protein, 42 aa, synthetic construct
  REGION 1..42: note = Synthetic: sUb-PGG mask
GIPPDQQRLIFAGKQLEDGRTLSDYNIQKESTLHLVLRLPGG

SEQ ID NO: 67 - protein, 39 aa, synthetic construct
  REGION 1..39: note = Synthetic: sUb mask 1
GIPPDQQRLIFAGKQLEDGRTLSDYNIQKESTLHLVLRL

SEQ ID NO: 68 - protein, 37 aa, synthetic construct; identical to SEQ ID NO: 2
  REGION 1..37: note = Synthetic: sUbmask 2

SEQ ID NO: 69 - protein, 43 aa, synthetic construct
  REGION 1..43: note = Synthetic: sUb-extmask
MQIFVKTLTGKTITLEVEPSDTIENVKAKIQDKEGIPPDQQRL

SEQ ID NO: 70 - protein, 37 aa, synthetic construct
  REGION 1..37: note = Synthetic: sUb-short mask
GIPPDQQRLIFAGKQLEDGRTLSDYNIQKESTLHLVL

SEQ ID NO: 71 - protein, 39 aa, synthetic construct
  REGION 1..39: note = Synthetic: sUb-flip mask
MQIFVKTLTGKTITLEVEPSDTIENVKAKIQDKEGIPGG

SEQ ID NO: 72 - protein, 37 aa, synthetic construct
  REGION 1..37: note = Synthetic: sUb-Q2Kmask
MKIFVKTLTGKTITLEVEPSDTIENVKAKIQDKEGIP

SEQ ID NO: 73 - protein, 37 aa, synthetic construct
  REGION 1..37: note = Synthetic: sUb-Q2Rmask
MRIFVKTLTGKTITLEVEPSDTIENVKAKIQDKEGIP

SEQ ID NO: 74 - protein, 37 aa, synthetic construct
  REGION 1..37: note = Synthetic: sUb-K11C- mask
MQIFVKTLTGCTITLEVEPSDTIENVKAKIQDKEGIP

SEQ ID NO: 75 - protein, 37 aa, synthetic construct
  REGION 1..37: note = Synthetic: sUb-P19Cmask
MQIFVKTLTGKTITLEVECSDTIENVKAKIQDKEGIP

SEQ ID NO: 76 - protein, 37 aa, synthetic construct
  REGION 1..37: note = Synthetic: sUb-Q31Kmask
MQIFVKTLTGKTITLEVEPSDTIENVKAKIKDKEGIP

SEQ ID NO: 77 - protein, 37 aa, synthetic construct
  REGION 1..37: note = Synthetic: sUb-Q31Rmask
MQIFVKTLTGKTITLEVEPSDTIENVKAKIRDKEGIP

SEQ ID NO: 78 - protein, 37 aa, synthetic construct
  REGION 1..37: note = Synthetic: sUb-Q31Cmask
MQIFVKTLTGKTITLEVEPSDTIENVKAKICDKEGIP

SEQ ID NO: 79 - protein, 37 aa, synthetic construct
  REGION 1..37: note = Synthetic: sUb-F4Cmask
MQICVKTLTGKTITLEVEPSDTIENVKAKIQDKEGIP

SEQ ID NO: 80 - protein, 37 aa, synthetic construct
  REGION 1..37: note = Synthetic: sUb-F4Emask
MQIEVKTLTGKTITLEVEPSDTIENVKAKIQDKEGIP

SEQ ID NO: 81 - protein, 37 aa, synthetic construct
  REGION 1..37: note = Synthetic: sUb-F4D mask
MQIDVKTLTGKTITLEVEPSDTIENVKAKIQDKEGIP

SEQ ID NO: 82 - protein, 37 aa, synthetic construct
  REGION 1..37: note = Synthetic: sUb-Q2K-V26F mask
MKIFVKTLTGKTITLEVEPSDTIENFKAKIQDKEGIP

SEQ ID NO: 83 - protein, 37 aa, synthetic construct
  REGION 1..37: note = Synthetic: sUb-Q2K-V5L-V26L mask
MKIFLKTLTGKTITLEVEPSDTIENLKAKIQDKEGIP

SEQ ID NO: 84 - protein, 41 aa, synthetic construct
  REGION 1..41: note = Synthetic: sUb-E34D mask
GDGIPPDQQRLIFAGKQLEDGRTLSDYNIQKESTLHLVLRL

SEQ ID NO: 85 - protein, 41 aa, synthetic construct
  REGION 1..41: note = Synthetic: sUb-E34C mask
GCGIPPDQQRLIFAGKQLEDGRTLSDYNIQKESTLHLVLRL

SEQ ID NO: 86 - protein, 39 aa, synthetic construct
  REGION 1..39: note = Synthetic: sUb-S57C mask
GIPPDQQRLIFAGKQLEDGRTLCDYNIQKESTLHLVLRL

SEQ ID NO: 87 - protein, 39 aa, synthetic construct
  REGION 1..39: note = Synthetic: sUb-P38D mask
GIPDDQQRLIFAGKQLEDGRTLSDYNIQKESTLHLVLRL

SEQ ID NO: 88 - protein, 39 aa, synthetic construct
  REGION 1..39: note = Synthetic: sUb-P38C mask
GIPCDQQRLIFAGKQLEDGRTLSDYNIQKESTLHLVLRL

SEQ ID NO: 89 - protein, 39 aa, synthetic construct
  REGION 1..39: note = Synthetic: sUb-P38E mask
GIPEDQQRLIFAGKQLEDGRTLSDYNIQKESTLHLVLRL

SEQ ID NO: 90 - protein, 39 aa, synthetic construct
  REGION 1..39: note = Synthetic: sUb-T66C mask
GIPPDQQRLIFAGKQLEDGRTLSDYNIQKESCLHLVLRL

SEQ ID NO: 91 - protein, 39 aa, synthetic construct
  REGION 1..39: note = Synthetic: sUb-T66K mask
GIPPDQQRLIFAGKQLEDGRTLSDYNIQKESKLHLVLRL

SEQ ID NO: 92 - protein, 39 aa, synthetic construct
  REGION 1..39: note = Synthetic: sUb-T66R mask
GIPPDQQRLIFAGKQLEDGRTLSDYNIQKESRLHLVLRL

SEQ ID NO: 93 - protein, 39 aa, synthetic construct
  REGION 1..39: note = Synthetic: sUb-T66K-L56K mask
GIPPDQQRLIFAGKQLEDGRTKSDYNIQKESKLHLVLRL

SEQ ID NO: 94 - protein, 39 aa, synthetic construct
  REGION 1..39: note = Synthetic: sUb-L43F mask
GIPPDQQRFIFAGKQLEDGRTLSDYNIQKESTLHLVLRL

(19) United States
(12) Patent Application Publication
(10) Pub. No.: US 2024/0352080 A1
(43) Pub. Date: Oct. 24, 2024

(54) Antibody Masking Domains

(71) Applicant: Seagen Inc., Bothell, WA (US)

(72) Inventor: Vivian Trang, Lynnwood, WA (US)

(73) Assignee: Seagen Inc., Bothell, WA (US)

(21) Appl. No.: 18/577,746

(22) PCT Filed: Jul. 13, 2022
(86) PCT No.: PCT/US2022/073662

§ 371 Date: Jan. 9, 2024

Related U.S. Application Data
(60) Provisional application No. 63/221,619, filed on Jul. 14, 2021.

(51) Int. Cl. C07K 14/47; A61K 47/64; A61K 47/68; A61P 35/00; C07K 16/22; C07K 16/28; C07K 16/30

(52) U.S. Cl. CPC C07K 14/4702; A61K 47/64; A61K 47/6803; A61K 47/68031; A61K 47/68033; A61K 47/68035; A61K 47/68037; A61K 47/6805; A61K 47/6809; A61K 47/6845; A61K 47/6849; A61K 47/6851; A61P 35/00; C07K 16/22; C07K 16/2803; C07K 16/2827; C07K 16/2863; C07K 16/2866; C07K 16/2875; C07K 16/2878; C07K 16/2896; C07K 16/3084; C07K 2319/30; C07K 2319/50

(57) ABSTRACT

Masking domains and masked antibodies comprising masking domains are provided. In some embodiments, masked antibodies comprising the masking domains demonstrate minimal aggregation and low potential for immunogenicity. In various embodiments, masked antibodies comprising the masking domains may be used in therapeutic treatment.

### CROSS REFERENCE TO RELATED APPLICATIONS

This application claims the benefit of priority of U.S. Provisional Application No. 63/221,619, filed Jul. 14, 2021, which is incorporated by reference in its entirety for any purpose.

### FIELD OF THE INVENTION

The present invention relates to the field of masked antibodies comprising ubiquitin masking domains. In particular, the present invention relates to masked antibodies with altered properties, such as minimal aggregation and low potential for immunogenicity of the masked antibody.

### BACKGROUND

Current antibody-based therapeutics may have less than optimal selectivity for the intended target. Although monoclonal antibodies are typically specific for binding to their intended targets, most target molecules are not specific to the disease site and may be present in cells or tissues other than the disease site.

Several approaches have been described for overcoming these off-target effects by engineering antibodies to have a cleavable linker attached to an inhibitory or masking domain that inhibits antibody binding (see, e.g., WO2003/068934, WO2004/009638, WO 2009/025846, WO2101/081173 and WO2014103973). The linker can be designed to be cleaved by enzymes that are specific to certain tissues or pathologies, thus enabling the antibody to be preferentially activated in desired locations. Masking moieties can act by binding directly to the binding site of an antibody or can act indirectly via steric hindrance. Various masking moieties, linkers, protease sites and formats of assembly have been proposed. The extent of masking may vary between different formats as may the compatibility of masking moieties with expression, purification, conjugation, or pharmacokinetics of antibodies.

### SUMMARY

The present disclosure relates to masked antibodies that comprise a removable masking agent (e.g., a ubiquitin masking agent) that prevents binding of the antibodies to their intended targets until the masking agent is cleaved off or otherwise removed. In other words, the masking agent masks the antigen binding portion of the antibody so that it cannot interact with its targets. In certain therapeutic uses, the masking agent can be removed (e.g., cleaved) by one or more molecules (e.g., proteases) that are present in an in vivo environment after administration of the masked antibody to a patient. In other uses, for example, non-therapeutic uses, a masking agent could be removed by adding one or more proteases to the medium in which the antibody is being used. Removal of the masking agent restores the ability of the antibodies to bind to their targets, thus enabling specific targeting of the antibodies.

The following non-limiting embodiments are provided.

Embodiment 1. A masked antibody or antigen-binding fragment thereof comprising a first masking domain and a second masking domain, wherein the first masking domain comprises a portion of a ubiquitin or ubiquitin variant fused to a heavy chain variable region (VH) of an antibody, and the second masking domain comprises a portion of a ubiquitin or ubiquitin variant fused to a light chain variable region (VL) of the antibody.

Embodiment 2. The masked antibody or antigen-binding fragment of Embodiment 1, comprising an N-terminal portion of a ubiquitin or ubiquitin variant and a C-terminal portion of a ubiquitin or ubiquitin variant.

Embodiment 3. The masked antibody or antigen-binding fragment of Embodiment 2, wherein the N-terminal portion of the ubiquitin or ubiquitin variant and the C-terminal portion of the ubiquitin or ubiquitin variant have an overlap of 0-10 amino acids, such as 0 amino acids, 1 amino acid, 2 amino acids, 3 amino acids, 4 amino acids, 5 amino acids, 6 amino acids, 7 amino acids, 8 amino acids, 9 amino acids, or 10 amino acids compared to a full-length ubiquitin.

Embodiment 4. The masked antibody or antigen-binding fragment of any of the preceding Embodiments, wherein the N-terminus of the VH is fused to a C-terminal portion of a ubiquitin or ubiquitin variant, and the N-terminus of the VL is fused to a N-terminal portion of a ubiquitin or ubiquitin variant.

Embodiment 5. The masked antibody or antigen-binding fragment of any of Embodiments 1 to 3, wherein the N-terminus of the VH is fused to a N-terminal portion of a ubiquitin or ubiquitin variant, and the N-terminus of the VL is fused to a C-terminal portion of a ubiquitin or ubiquitin variant.

Embodiment 6. The masked antibody or antigen-binding fragment of any one of the preceding Embodiments, wherein the N-terminal portion of the ubiquitin or ubiquitin variant and the C-terminal portion of the ubiquitin or ubiquitin variant block the binding of the antigen-binding domain to its antigen.

Embodiment 7. The masked antibody or antigen-binding fragment of any one of the preceding Embodiments, wherein the N-terminal portion of the ubiquitin variant comprises one or more amino acid substitutions and/or a C-terminal extension compared to the corresponding portion of a wild-type ubiquitin.

Embodiment 8. The masked antibody or antigen-binding fragment of any one of the preceding Embodiments, wherein the N-terminal portion of the ubiquitin or ubiquitin variant comprises at least 30 amino acid residues, such as at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 amino acid residues.

Embodiment 9. The masked antibody or antigen-binding fragment of any one of the preceding Embodiments, wherein the N-terminal portion of the ubiquitin or ubiquitin variant comprises amino acids 1-37 of SEQ ID NO: 1.

Embodiment 10. The masked antibody or antigen-binding fragment of any one of the preceding Embodiments, wherein the C-terminal portion of the ubiquitin or ubiquitin variant comprises at least 30 amino acid residues, such as at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 amino acid residues.

Embodiment 11. The masked antibody or antigen-binding fragment of any one of the preceding Embodiments, wherein the C-terminal portion of the ubiquitin or ubiquitin variant comprises amino acids 35-76 of SEQ ID NO:1, optionally wherein a C-terminal fragment, such as 1 amino acid, 2 amino acids, 3 amino acids, 4 amino acids, 5 amino acids, or 6 amino acids, is removed.

Embodiment 12. The masked antibody or antigen-binding fragment of any one of the preceding Embodiments, wherein the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 1, and wherein the variant comprises a modification at one or more positions corresponding to positions 2, 4, 5, 11, 19, 26, 31, 34, 38, 43, 56, 57, 64, 66, 74, 75, 76 of SEQ ID NO: 1, preferably wherein the modification is a substitution.

Embodiment 13. The masked antibody or antigen-binding fragment of any one of the preceding Embodiments, wherein the first masking domain and the second masking domain comprise one or more interacting surfaces.

Embodiment 14. The masked antibody or antigen-binding fragment of Embodiment 13, wherein the ubiquitin variant comprises one or more substitutions at one or more interacting surfaces of the first masking domain and the second masking domain.

Embodiment 15. The masked antibody or antigen-binding fragment of any one of the preceding Embodiments, wherein the first masking domain and the second masking domain interact to form a globular structure.

Embodiment 16. The masked antibody or antigen-binding fragment of any one of the preceding Embodiments, wherein the ubiquitin variant comprises one or more substitutions at a position corresponding to position Q2; positions Q2 and E64; positions F4 and T66; positions K11 and E34; positions P19 and S57; positions Q31 and P38; position L56; and/or position T66 of SEQ ID NO: 1.

Embodiment 17. The masked antibody or antigen-binding fragment of any one of the preceding Embodiments, wherein the ubiquitin variant comprises one or more of, such as two or more of, three or more of, four or more of, or all five of the following pairs of substitutions: Q2C and E64C; F4C and T66C; K11C and E34C; P19C and S57C; and/or Q31C and P38C of SEQ ID NO: 1.

Embodiment 18. The masked antibody or antigen-binding fragment of any one of Embodiments 1 to 16, wherein the ubiquitin variant comprises a substitution Q2D/E/K/R; F4D/E/K/R; K11D/E/R; P19D/E/K/R; Q31D/E/K/R; E34D/K/R; P38D/E/K/R; S57D/E/K/R; and/or T66D/E/K/R of SEQ ID NO: 1.

Embodiment 19. The masked antibody or antigen-binding fragment of any one of Embodiments 1 to 16 or 18, wherein the ubiquitin variant comprises a substitution Q2K/R; a substitution E34D; a substitution L43F; a substitution L56K; a substitution T66K; and/or comprises a pair of substitutions F4D/E and T66K/R; K11R and E34D; and/or Q31R and P38D of SEQ ID NO: 1.

Embodiment 20. The masked antibody or antigen-binding fragment of any one of Embodiments 1 to 16 or 18-19, wherein the ubiquitin variant comprises a substitution Q2D/E/K/R, such as Q2K or Q2R of SEQ ID NO: 1.

Embodiment 21. The masked antibody or antigen-binding fragment of any one of the preceding Embodiments, wherein the N-terminal portion of the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 2, and wherein the variant comprises a modification at one or more positions corresponding to positions 2, 4, 5, 11, 19, 26, 31, of SEQ ID NO: 2, preferably wherein the modification is a substitution.

Embodiment 22. The masked antibody or antigen-binding fragment of any one of the preceding Embodiments, wherein the ubiquitin variant comprises a substitution V5L of SEQ ID NO: 1 or SEQ ID NO: 2.

Embodiment 23. The masked antibody or antigen-binding fragment of any one of the preceding Embodiments, wherein the ubiquitin variant comprises a substitution V26F/L of SEQ ID NO: 1 or SEQ ID NO: 2.

Embodiment 24. The masked antibody or antigen-binding fragment of any one of the preceding Embodiments, wherein the C-terminal portion of the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 3, and wherein the variant comprises a modification at one or more positions corresponding to positions 1, 4, 9, 40, 41, 42 SEQ ID NO: 3, preferably wherein the modification is a substitution.

Embodiment 25. The masked antibody or antigen-binding fragment of any one of the preceding Embodiments, wherein the ubiquitin variant comprises a substitution L9F of SEQ ID NO: 3.

Embodiment 26. The masked antibody or antigen-binding fragment of any one of the preceding Embodiments, wherein the ubiquitin variant comprises a substitution R40P of SEQ ID NO: 3.

Embodiment 27. The masked antibody or antigen-binding fragment of any one of the preceding Embodiments, wherein the C-terminus of SEQ ID NO: 1, SEQ ID NO: 2 and/or SEQ ID NO: 3 comprises a linker, e.g., of the form S(G)nS, wherein n is from 5-20; or of the form (G)n, wherein n is an integer of at least one, such as Gly-Gly; or of the form (GS)n, wherein n is an integer of at least one, such as Gly-Ser; such as a linker of SEQ ID NOs: 44-55, such as SEQ ID NO: 44 or SEQ ID NO: 50.

Embodiment 28. The masked antibody or antigen-binding fragment of any one of the preceding Embodiments, wherein the N-terminal portion of the ubiquitin or ubiquitin variant has at least 90% sequence identity, such as at least 92%, at least 93%, at least 94%, at least 95%, at least 96%, at least 97%, at least 98%, at least 99% or 100% sequence identity to any one of SEQ ID NOs: 2, 6-10, 18-32, 66-94.

Embodiment 29. The masked antibody or antigen-binding fragment of any one of the preceding Embodiments, wherein the N-terminal portion of the ubiquitin or ubiquitin variant comprises or consists of any one of SEQ ID NOs: 2, 6-10, 18-32, 66-94.

Embodiment 30. The masked antibody or antigen-binding fragment of any one of the preceding Embodiments, wherein the C-terminal portion of the ubiquitin or ubiquitin variant has at least 90% sequence identity, such as at least 92%, at least 93%, at least 94%, at least 95%, at least 96%, at least 97%, at least 98%, at least 99% or 100% sequence identity to any one of SEQ ID NOs: 3-5, 11-17, 33-43, 66-94.

Embodiment 31. The masked antibody or antigen-binding fragment of any one of the preceding Embodiments, wherein the C-terminal portion of the ubiquitin or ubiquitin variant comprises or consists of any one of SEQ ID NOs: 3-5, 11-17, 33-43, 66-94.

Embodiment 32. The masked antibody or antigen-binding fragment of any one of the preceding Embodiments, wherein the first masking domain and the second masking domain comprise the sequences of SEQ ID NOs: 2 and 4, respectively; or SEQ ID NOs: 2 and 5, respectively; or SEQ ID NOs: 6 and 12, respectively; or SEQ ID NOs: 7 and 13, respectively; or SEQ ID NOs: 8 and 14, respectively; or SEQ ID NOs: 9 and 15, respectively, or SEQ ID NOs: 10 and 16, respectively, or SEQ ID NOs: 7 and 17, respectively; or SEQ ID NOs: 11 and 18, respectively; or SEQ ID NOs: 19 and 12, respectively; or SEQ ID NOs: 20 and 13, respectively; or SEQ ID NOs: 21 and 14, respectively; or SEQ ID NOs: 22 and 12, respectively; SEQ ID NOs: 23 and 34, respectively; or SEQ ID NOs: 24 and 35, respectively; or SEQ ID NOs: 25 and 36, respectively; or SEQ ID NOs: 26 and 36, respectively; or SEQ ID NOs: 25 and 38, respectively; or SEQ ID NOs: 26 and 38, respectively; or SEQ ID NOs: 27 and 37, respectively; or SEQ ID NOs: 28 and 39, respectively; or SEQ ID NOs: 29 and 40, respectively; or SEQ ID NOs: 29 and 41, respectively; or SEQ ID NOs: 30 and 40, respectively; or SEQ ID NOs: 30 and 41, respectively; or SEQ ID NOs: 31 and 13, respectively; or SEQ ID NOs: 32 and 13, respectively; or SEQ ID NOs: 6 and 33, respectively; or SEQ ID NOs: 6 and 42, respectively; or SEQ ID NOs: 7 and 43, respectively.

Embodiment 33. The masked antibody or antigen-binding fragment of any one of the preceding Embodiments, wherein each of the first masking domain and the second masking domain comprises a protease-cleavable linker and is linked to the heavy chain and the light chain via the protease-cleavable linker.

Embodiment 34. The masked antibody or antigen-binding fragment of Embodiment 33, wherein the protease-cleavable linker of the first masking domain and the protease-cleavable linker of the second masking domain are the same.

Embodiment 35. The masked antibody or antigen-binding fragment of Embodiment 33 or Embodiment 34, wherein the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site.

Embodiment 36. The masked antibody or antigen-binding fragment of any one of Embodiments 33-35, wherein the protease-cleavable linker comprises a matrix metalloprotease (MMP) cleavable sequence.

Embodiment 37. The masked antibody or antigen-binding fragment of Embodiment 36, wherein the MMP cleavage site is selected from an MMP2 cleavage site, an MMP7 cleavage site, an MMP9 cleavage site and an MMP13 cleavage site.

Embodiment 38. The masked antibody or antigen-binding fragment of Embodiment 36 or Embodiment 37, wherein the MMP cleavage site comprises the sequence IPVSLRSG (SEQ ID NO: 57) or GPLGVR (SEQ ID NO: 59).

Embodiment 39. The masked antibody or antigen-binding fragment of any one of Embodiments 36-38, wherein following cleavage by an MMP, the heavy chain and/or light chain of the antibody or antigen-binding fragment comprises a stub amino acid remnant of the MMP cleavage site.

Embodiment 40. The masked antibody or antigen-binding fragment of Embodiment 39, wherein the stub amino acid remnant comprises the sequence LRSG (SEQ ID NO: 65), SG, or VR at the N terminus of the antibody.

Embodiment 41. The masked antibody or antigen-binding fragment of any of Embodiments 1 to 32, wherein each of the first masking domain and the second masking domain comprises a protease-resistant linker and is linked to the heavy chain and/or the light chain via the protease-resistant linker.

Embodiment 42. The masked antibody or antigen-binding fragment of Embodiment 41, wherein the protease-resistant linker comprises LALGPG (SEQ ID NO: 63).

Embodiment 43. The masked antibody or antigen-binding fragment of any one of the preceding Embodiments, wherein the antibody or antigen-binding fragment binds a therapeutic antigen.

Embodiment 44. The masked antibody or antigen-binding fragment of Embodiment 43, wherein the antibody is useful for treating cancer, an autoimmune disorder, or an infection.

Embodiment 45. The masked antibody or antigen-binding fragment of Embodiment 43, wherein the antibody binds a tumor-associated antigen.

Embodiment 46. The masked antibody or antigen-binding fragment of Embodiment 45, wherein the tumor-associated antigen is selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Axl, Clec12A, CD352, FAP, CXCR3, and CD5.

Embodiment 47. The masked antibody or antigen-binding fragment of any one of Embodiments 1-46, wherein the masked antibody has reduced binding affinity for the antibody's target antigen compared to the binding affinity of the antibody for its target antigen without the first masking domain and second masking domain.

Embodiment 48. The masked antibody or antigen-binding fragment of Embodiment 47, wherein the binding affinity is reduced at least about 30-fold compared to the antibody or antigen-binding fragment without the first masking domain and the second masking domain.

Embodiment 49. The masked antibody or antigen-binding fragment of Embodiment 47 or Embodiment 48, wherein the binding affinity is reduced at least about 50-fold compared to the antibody or antigen-binding fragment without the first masking domain and the second masking domain.

Embodiment 50. The masked antibody or antigen-binding fragment of any one of Embodiments 47-49, wherein the binding affinity is reduced at least about 80-fold compared to the antibody or antigen-binding fragment without the first masking domain and the second masking domain.

Embodiment 51. The masked antibody or antigen-binding fragment of any one of Embodiments 47-50, wherein the binding affinity is reduced at least about 100-fold compared to the antibody or antigen-binding fragment without the first masking domain and the second masking domain.

Embodiment 52. An immunoconjugate comprising the masked antibody or antigen-binding fragment of any one of Embodiments 1-51 and a cytotoxic agent.

Embodiment 53. The immunoconjugate of Embodiment 52, wherein the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer.

Embodiment 54. The immunoconjugate of Embodiment 52 or Embodiment 53, wherein the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid.

Embodiment 55. The immunoconjugate of any one of Embodiments 52-54, wherein the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine.

Embodiment 56. The immunoconjugate of Embodiment 55, wherein the cytotoxic agent is an auristatin.

Embodiment 57. The immunoconjugate of Embodiment 55, wherein the cytotoxic agent is MMAE or MMAF.

Embodiment 58. A nucleic acid sequence encoding the masked antibody or antigen-binding fragment of any one of Embodiments 1-51.

Embodiment 59. An expression vector comprising the nucleic acid of Embodiment 58.

Embodiment 60. A host cell comprising the nucleic acid of Embodiment 58 or the expression vector of Embodiment 59.

Embodiment 61. A host cell that expresses the masked antibody or antigen-binding fragment of any one of Embodiments 1-51.

Embodiment 62. A method of producing the masked antibody or antigen-binding fragment of any one of Embodiments 1-51 comprising culturing the host cell of Embodiment 60 or Embodiment 61.

Embodiment 63. The method of Embodiment 62, further comprising isolating the masked antibody or antigen-binding fragment thereof.

Embodiment 64. A pharmaceutical composition comprising the masked antibody or antigen-binding fragment of any one of Embodiments 1-51 or the immunoconjugate of any one of Embodiments 52-57 and a pharmaceutically acceptable carrier.

Embodiment 65. The masked antibody or antigen-binding fragment of any one of Embodiments 1-51, the immunoconjugate of any one of Embodiments 52-57, or the pharmaceutical composition of Embodiment 64 for use in therapy.

Embodiment 66. The masked antibody or antigen-binding fragment of any one of Embodiments 1-51, the immunoconjugate of any one of Embodiments 52-57, or the pharmaceutical composition of Embodiment 64 for use in treating cancer, an autoimmune disease, or an infection.

Embodiment 67. Use of the masked antibody or antigen-binding fragment of any one of Embodiments 1-51, the immunoconjugate of any one of Embodiments 52-57, or the pharmaceutical composition of Embodiment 64 for the preparation of a medicament for use in therapy.

Embodiment 68. Use of the masked antibody or antigen-binding fragment of any one of Embodiments 1-51, the immunoconjugate of any one of Embodiments 52-57, or the pharmaceutical composition of Embodiment 64 for the preparation of a medicament for use in treating cancer, an autoimmune disease, or an infection.

Embodiment 69. A method comprising administering to a subject in need thereof the masked antibody or antigen-binding fragment of any one of Embodiments 1-51, the immunoconjugate of any one of Embodiments 52-57, or the pharmaceutical composition of Embodiment 64.

Embodiment 70. A method of treating cancer, an autoimmune disease, or an infection comprising administering to a subject in need thereof the masked antibody or antigen-binding fragment of any one of Embodiments 1-51, the immunoconjugate of any one of Embodiments 52-57, or the pharmaceutical composition of Embodiment 64.

Embodiment 71. The method of Embodiment 70, which is a method of treating cancer.

Embodiment 72. The method of Embodiment 71, wherein the cancer is a solid cancer, a soft tissue cancer, or a hematopoietic cancer.

Embodiment 73. The method of Embodiment 71 or Embodiment 72, wherein the cancer is selected from lung cancer, pancreatic cancer, breast cancer, liver cancer, ovarian cancer, testicular cancer, kidney cancer, bladder cancer, spinal cancer, brain cancer, cervical cancer, endometrial cancer, colorectal cancer, anal cancer, endometrial cancer, esophageal cancer, gallbladder cancer, gastrointestinal cancer, gastric cancer, sarcoma, head and neck cancer, melanoma, skin cancer, prostate cancer, pituitary cancer, stomach cancer, uterine cancer, vaginal cancer, thyroid cancer, a sarcoma, soft tissue sarcoma, osteosarcoma, a lymphoma, diffuse large B-cell lymphomas (DLBCL), follicular lymphoma, myelodysplastic syndrome (MDS), Hodgkin's disease, a malignant lymphoma, non-Hodgkin's lymphoma, Burkitt's lymphoma, multiple myeloma, Richter's Syndrome, a leukemia, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMoL).

### BRIEF DESCRIPTION OF THE DRAWINGS

FIG. 1 shows a split ubiquitin blocking strategy. Ubiquitin is split in two at the loop connecting the alpha helix and beta strand. Positions 35-37 are repeated in both the heavy and light chain halves to provide overlap.

FIG. 2 provides analytical size-exclusion chromatography (SEC) data for the percentage high molecular weight (%) of Antibody A-low affinity antibody at time 0 (FIG. 2A) hours and in a time course assessment over 37 days at room temperature (FIG. 2B).

FIG. 3 provides reverse-phase liquid chromatography-mass spectrometry (LC-MS) data for alternative C-termini of the mask. Masks having a “PGG” moiety resulted in premature cleavage of the mask on the heavy chain during expression (FIG. 3A) whereas masks having a C-terminal “GS” linker successfully mitigated the cleavage event (FIG. 3B).

FIG. 4 shows binding experiments for anti-Antigen 1 (unmasked parent) or anti-hB6H12.3 (unmasked parent) as well as their masked counterparts having coiled-coil mask (as described in, e.g., Trang et al., Nature Biotech. 37, 761-765 (2019)) or ubiquitin mask (having light chain mask of SEQ ID NO: 2 and heavy chain mask of SEQ ID NO: 4). FIG. 4A shows saturation binding flow cytometry results on Antigen-1-expressing Ramos cells via competition with AlexaFluor 647-labeled Anti-Antigen-1. FIG. 4B shows saturation binding flow cytometry results.

FIG. 5 shows saturation binding FACS results with Antibody A target-expressing HEK293F cells to assess the impact of the linker sequence using variants as in Tables 3 and 4. Changing the linker sequence can improve the blocking ability of the sUb-mask.

FIG. 6 shows saturation binding flow cytometry results for anti-hB6H12.3 (unmasked parent) and ubiquitin mask having light chain mask of SEQ ID NO: 2 and heavy chain mask of SEQ ID NO: 4 (FIG. 6A), as well as the sUbext variants as in Tables 3 and 4 (FIG. 6B).

FIG. 7A-7B show the structure of ubiquitin. To further improve the blocking ability of a split ubiquitin mask, point mutations were made along interacting surfaces of the two halves of ubiquitin based on the crystal structure of the protein (FIG. 7A-7B).

FIG. 8 shows saturation binding flow cytometry results of unmasked parent and several variants as in Tables 5 and 6, having point mutations to create disulfide bonds between the ubiquitin halves.

FIG. 9A-9C shows saturation binding flow cytometry results on various cell types of unmasked parent and several variants as in Tables 5 and 6, having point mutations to create salt bridges between the ubiquitin halves.

FIG. 10A-10C shows in vitro results to compare the change in cytotoxicity of Antibody A low and high affinity antibody-drug conjugate (ADC) against ubiquitin mask low affinity ADC (having light chain mask of SEQ ID NO: 6 and heavy chain mask of SEQ ID NO: 12) on various cell lines.

FIG. 11 shows saturation binding flow cytometry results comparing h15H3-low and high affinity ADC parents against ubiquitin masked low affinity ADC (having light chain mask of SEQ ID NO: 6 and heavy chain mask of SEQ ID NO: 12) on various cell lines.

FIG. 12A-12B shows an assessment of in vivo stability of a sUb mask in nude mice. Plasma was assessed via western blot at day 1 and day 4. By day 4, the majority of C-terminal half of the sUb mask was removed on the heavy chain. To assess the ubiquitination state of the masked antibody, the western blot was further probed using anti-ubiquitin. The ubiquitin mask does not serve as a substrate for further ubiquitination given the lack of higher molecular weight species on the heavy and light chain of the antibody as shown by the anti hFc and anti-ubiquitin blot.

FIG. 13 shows the activity of ubiquitin masked vs unmasked ADCs of low affinity Antibody A in a pancreatic cancer xenograft tumor model in Nude mice. Despite having a ˜20-fold difference in activity in vitro, in vivo, the masked antibody has approximately 3-fold less activity than the unmasked ADC, which suggests that the mask is cleaved from the ADC leading to binding and uptake of the cell.

FIG. 14A-FIG. 14B shows the activity of ubiquitin masked vs unmasked ADCs of low affinity h15H3 in a pancreatic cancer xenograft tumor models in Nude mice. In the HPAF-II model, the masked ADC displays anti-tumor activity, but not to the same extent as the unmasked ADC (FIG. 14A). The decrease in activity may be attributed to the cleavage kinetics of the mask on h15H3-low. In the BxPC3 model, sUb h15H3-low displays similar anti-tumor activity to the unmasked ADC (FIG. 14B).

FIG. 15A-FIG. 15D shows an evaluation of on-target toxicity in masked vs unmasked ADCs. At 6 mg/kg in Sprague-Dawley rats, unmasked h15H3-low ADCs have moderate skin toxicities that result in increased mitotic/apoptopic figures and epidermal thickening. These events can be found in both mammary and neck regions. Administration of h15H3-low gluc-AT at 6 mg/kg resulted in mild skin changes with the score of 0-1 where there is minimal increase in mitotic/apoptotic figures. These results suggest that the masking strategy is able to reduce the on-target toxicity found in the rodent cross-reactive h15H3-low gluc-MMAE ADC.

### DETAILED DESCRIPTION

The invention provides masking domains that may be used in masked antibodies. In the masked antibody, the variable regions are masked by linkage of the variable region chains to a portion of a ubiquitin protein. The portions of a ubiquitin protein associate with one another (i.e., the ubiquitin protein maintains its globular structure despite having two ends appended to an antibody) and, in some embodiments, sterically inhibit binding of the antibody binding site to its target. The portion of a ubiquitin protein may be linked to the heavy chain and light chain variable regions of the antibody. Masking of antibodies by this format can reduce binding affinities (and cytotoxic activities in the case of ADCs) by over thirty-fold or by over fifty-fold, and in some embodiments, can reduce off-target effects. In some embodiments, the masking domains described herein provide altered properties of masked antibodies, such as minimal aggregation and low potential for immunogenicity.

In certain exemplary embodiments, antibodies are provided that comprise a removable mask (e.g., a mask comprising a ubiquitin domain) that blocks binding of the antibody to its antigenic target. In certain embodiments, masking ubiquitin domain is attached to the amino-terminus of one or more of the heavy and/or light chains of the antibody via a matrix metalloproteinase (MMP)-cleavable linker sequence. In a tumor microenvironment, for example, altered proteolysis leads to unregulated tumor growth, tissue remodeling, inflammation, tissue invasion, and metastasis. See, e.g., Kessenbrock (2011) Cell 141:52. MMPs represent the most prominent family of proteinases associated with tumorigenesis, and MMPs mediate many of the changes in the microenvironment during tumor progression. Id. Upon exposure of the antibody of the present invention to an MMP, the MMP linker sequence is cleaved, thus allowing removal of the ubiquitin mask and enabling the antibody to bind its target antigen in a tumor microenvironment-specific manner.

In other embodiments, such as for use in vitro, such as in medical diagnostics, chemical processing, or industrial uses, masked antibodies may be useful so that antibody activity can be controlled by addition of an exogenous protease to the solution at an appropriate point to cleave off the ubiquitin domain of the mask and allow the antibodies to bind to their targets.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

Compositions or methods “comprising” one or more recited elements or steps may include other elements or steps not specifically recited. For example, a composition that comprises antibody may contain the antibody alone or in combination with other ingredients.

Compositions or methods “consisting essentially of” one or more steps may include elements or steps not specifically recited so long as any additional element or step does not materially alter the essential nature of the composition or method as recited in the claim. For example, other steps may be included so long as they do not materially alter the overall preparation process, such as wash steps or buffer changes.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and it also encompasses antigen-binding antibody fragments, such as a F(ab′)₂, a Fv fragment, a diabody, a single-chain antibody, an scFv fragment, or an scFv-Fc. Genetically engineered intact antibodies and fragments such as chimeric antibodies, humanized antibodies, single-chain Fv fragments, single-chain antibodies, diabodies, minibodies, linear antibodies, bispecific or bivalent, multivalent or multi-specific (e.g., bispecific) hybrid antibodies, and the like. Thus, the term “antibody” is used expansively to include any protein that comprises an antigen-binding site of an antibody and is capable of specifically binding to its antigen. In some embodiments, an antibody comprises two amino-termini (for example, comprises two polypeptide chains), such as a heavy chain (or fragment thereof) amino-terminus and a light chain (or fragment thereof) amino-terminus.

The term “antibody” includes a “naked” antibody that is not bound (i.e., covalently or non-covalently bound) to a masking compound of the invention. The term antibody also embraces a “masked” antibody, which comprises an antibody that is covalently or non-covalently bound to one or more masking compounds such as, e.g., a split ubiquitin protein, as described further herein. The term antibody includes a “conjugated” antibody or an “antibody-drug conjugate (ADC)” in which an antibody is covalently or non-covalently bound to a pharmaceutical agent, e.g., to a cytostatic or cytotoxic drug. In certain embodiments, an antibody is a naked antibody or antigen-binding fragment that optionally is conjugated to a pharmaceutical agent, e.g., to a cytostatic or cytotoxic drug. In other embodiments, an antibody is a masked antibody or antigen-binding fragment that optionally is conjugated to a pharmaceutical agent, e.g., to a cytostatic or cytotoxic drug.

Antibodies typically comprise a heavy chain variable region and a light chain variable region, each comprising three complementary determining regions (CDRs) with surrounding framework (FR) regions, for a total of six CDRs. An antibody light or heavy chain variable region (also referred to herein as a “light chain variable domain” (“VL domain”) or “heavy chain variable domain” (“VH domain”), respectively) comprises “framework” regions interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

Naturally occurring antibodies are usually tetrameric and consist of two identical pairs of heavy and light chains. In each pair, the light and heavy chain variable regions (VL and VH) are together primarily responsible for binding to an antigen, and the constant regions are primarily responsible for the antibody effector functions. Five classes of antibodies (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that comprises constant region protein domains (CH1, hinge, CH2, and CH3; IgG3 also contains a CH4 domain) that are substantially invariant for a given subclass in a species. Antibodies as defined herein, may include these natural forms as well as various antigen-binding fragments, as described above, antibodies with modified heavy chain constant regions, bispecific and multispecific antibodies, and masked antibodies.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

An “antigen-binding site” of an antibody is that portion of an antibody that is sufficient to bind to its antigen. The minimum such region is typically a fragment of a variable domain comprising six CDRs (or three CDRs in the case of a single-domain antibody). In some embodiments, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al, FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

Numbering of the heavy chain constant region is via the EU index as set forth in Kabat (Kabat, Sequences of Proteins of Immunological Interest, National Institutes of Health, Bethesda, MD, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

Although humanized antibodies often incorporate all six CDRs (preferably as defined by Kabat or IMGT®) from a mouse antibody, they can also be made with fewer than all six CDRs (e.g., at least 3, 4, or 5) from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs.

The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “therapeutic antigen” refers to an antigen that may be targeted by an antibody to achieve a beneficial therapeutic effect. In some embodiments, an antibody that binds to a therapeutic antigen may agonize, i.e., increase the activity of, the antigen. In some embodiments, an antibody that binds to a therapeutic antigen may antagonize, i.e., decrease the activity of, the antigen. In some embodiments, an antibody may bind a therapeutic antigen and achieve a beneficial effect by bringing another molecule or cell to the antigen (or to the cell that expresses the antigen). Nonlimiting examples of antibodies bringing another molecule or cell to the therapeutic antigen include, for example, an antibody-drug conjugate that brings a cytotoxic drug to a cell that expresses the therapeutic antigen; a bispecific antibody that brings a cytotoxic cell to the cell the expresses the therapeutic antigen (such as a bispecific antibody comprising an anti-CD3 binding domain, which recruits a cytotoxic T cell. For the avoidance of doubt, both antigens bound by a therapeutic bispecific antibody are considered therapeutic antigens.

An “antibody-drug conjugate” refers to an antibody conjugated to a cytotoxic agent or cytostatic agent. Typically, antibody-drug conjugates bind to a target antigen on a cell surface, followed by internalization of the antibody-drug conjugate into the cell and subsequent release of the drug into the cell.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The terms “patient” and “subject” refer to organisms to be treated by the methods described herein and includes human and other mammalian subjects such as non-human primates, mammals (e.g., murines, simians, equines, bovines, porcines, canines, felines, and the like), rabbits, rats, mice, and the like and transgenic species thereof, that receive either prophylactic or therapeutic treatment. In certain exemplary embodiments, a subject is a human patient suffering from or at risk of developing cancer, e.g., a solid tumor, that optionally secretes one or more proteases capable of cleaving a masking domain (e.g., a ubiquitin masking domain) of an antibody described herein.

As used herein, the terms, “treat,” “treatment” and “treating” includes any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

As used herein, the term “effective amount” refers to the amount of a compound (e.g., a masked antibody) sufficient to effect beneficial or desired results. An effective amount of an antibody is administered in an “effective regimen.” The term “effective regimen” refers to a combination of amount of the antibody being administered and dosage frequency adequate to accomplish prophylactic or therapeutic treatment of the disorder.

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an antibody is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, a “stub,” a “stub antibody” or a “stub antigen-binding fragment” refers to an antibody or antigen-binding fragment after cleavage by an MMP or an antibody or antigen-binding fragment that was generated recombinantly, i.e., produced without the ubiquitin masking domain and, therefore, no cleavage was performed. MIP cleavage leads to a short amino acid sequence remnant. For the IPV cleavage site, the remnant, or stub, sequence should be LRSG (SEQ ID NO: 65) or SG. For the M2 cleavage site, the remnant, or stub, sequence should be VR.

### I. Masking Domains Comprising a Portion of a Ubiquitin or Ubiquitin Variant

In certain embodiments, an antibody is associated with a masking domain comprising a portion of a ubiquitin or ubiquitin variant (also referred to as a “ubiquitin masking domain” or a “split ubiquitin masking domain”) that blocks binding of the antibody to its antigen target. In various embodiments, an antibody associated with a masking domain is referred to as a “masked antibody.”

Ubiquitin is a small protein that is found in almost all tissues in eukaryotic organisms and serves as a post-translational modification. While ubiquitin is usually found intracellularly, it is also present in human plasma and serum at nanomolar concentrations.

A split protein system derived from ubiquitin (split Ubiquitin or “sUb”) has been used as a sensor for protein-protein interactions which implies that the protein can maintain its globular structure despite having two ends appended to different proteins. Coupled with a low probability to cause an immune response, ubiquitin can provide an antibody masking strategy.

In some embodiments, a masking domain is provided that comprises a first masking domain and a second masking domain, wherein the first masking domain comprises a portion of a ubiquitin or ubiquitin variant. In some embodiments, a masking domain is provided that comprises a first masking domain and a second masking domain, wherein the second masking domain comprises a portion of a ubiquitin or ubiquitin variant. In some embodiments, a masking domain is provided that comprises a first masking domain and a second masking domain, wherein the first masking domain comprises a portion of a ubiquitin or ubiquitin variant and the second masking domain comprises a portion of a ubiquitin or ubiquitin variant.

In some embodiments, a masking domain is provided that comprises a first masking domain and a second masking domain, wherein the first masking domain comprises a portion of a ubiquitin or ubiquitin variant fused to a heavy chain variable region (VH) of an antibody, and the second masking domain comprises a portion of a ubiquitin or ubiquitin variant fused to a light chain variable region (VL) of the antibody.

In some embodiments, a masking domain is provided that comprises an N-terminal portion of a ubiquitin or ubiquitin variant. In some embodiments, a masking domain is provided that comprises a C-terminal portion of a ubiquitin or ubiquitin variant. In some embodiments, a masking domain is provided that comprises an N-terminal portion of a ubiquitin or ubiquitin variant and a C-terminal portion of a ubiquitin or ubiquitin variant.

In some embodiments, a ubiquitin masking domain is provided wherein the N-terminal portion of the ubiquitin or ubiquitin variant and the C-terminal portion of the ubiquitin or ubiquitin variant have an overlap of 0-10 amino acids, such as 0 amino acids, 1 amino acid, 2 amino acids, 3 amino acids, 4 amino acids, 5 amino acids, 6 amino acids, 7 amino acids, 8 amino acids, 9 amino acids, or 10 amino acids compared to a full-length ubiquitin.

Exemplary sequences shown below for light chains may be used with heavy chains and vice versa. Thus, in some embodiments, what is referred to as the “first” masking domain may be linked to the heavy chain or the light chain, and the “second” masking domain may be linked to the other chain. Thus, in some embodiments, the first masking domain is linked to the amino-terminus of the heavy chain and the second masking domain is linked to the amino-terminus of the light chain, and in some embodiments, the first masking domain is linked to the amino-terminus of the light chain and the second masking domain is linked to the amino-terminus of the heavy chain. Optionally, multiple copies of the portion of ubiquitin or ubiquitin variant are linked in tandem to the amino-termini of the heavy and light chains.

In some embodiments, a ubiquitin masking domain is provided wherein the N-terminus of the VH is fused to a C-terminal portion of a ubiquitin or ubiquitin variant, and the N-terminus of the VL is fused to a N-terminal portion of a ubiquitin or ubiquitin variant.

In some embodiments, a ubiquitin masking domain is provided wherein the N-terminus of the VH is fused to a N-terminal portion of a ubiquitin or ubiquitin variant, and the N-terminus of the VL is fused to a C-terminal portion of a ubiquitin or ubiquitin variant.

In some embodiments, a masking domain is provided wherein the N-terminal portion of the ubiquitin or ubiquitin variant and the C-terminal portion of the ubiquitin or ubiquitin variant block the binding of the antigen-binding domain of an antibody to its antigen.

In some embodiments, a ubiquitin masking domain is provided wherein the N-terminal portion of the ubiquitin variant comprises one or more amino acid substitutions compared to the corresponding portion of a wild-type ubiquitin. In some embodiments, a ubiquitin masking domain is provided wherein the N-terminal portion of the ubiquitin variant comprises a C-terminal extension compared to the corresponding portion of a wild-type ubiquitin. In some embodiments, a ubiquitin masking domain is provided wherein the N-terminal portion of the ubiquitin variant comprises one or more amino acid substitutions and/or a C-terminal extension compared to the corresponding portion of a wild-type ubiquitin.

In some embodiments, a ubiquitin masking domain is provided wherein the N-terminal portion of the ubiquitin or ubiquitin variant comprises at least 30 amino acid residues, such as at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 amino acid residues.

In some embodiments, a ubiquitin masking domain is provided wherein the N-terminal portion of the ubiquitin or ubiquitin variant comprises amino acids 1-37 of SEQ ID NO: 1.

In some embodiments, a ubiquitin masking domain is provided wherein the C-terminal portion of the ubiquitin or ubiquitin variant comprises at least 30 amino acid residues, such as at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 amino acid residues.

In some embodiments, a ubiquitin masking domain is provided wherein, the C-terminal portion of the ubiquitin or ubiquitin variant comprises amino acids 35-76 of SEQ ID NO: 1, optionally wherein a C-terminal fragment, such as 1 amino acid, 2 amino acids, 3 amino acids, 4 amino acids, 5 amino acids, or 6 amino acids, is removed.

In some embodiments, a ubiquitin masking domain is provided, wherein the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 1, and wherein the variant comprises a modification at one or more positions corresponding to positions 2, 4, 5, 11, 19, 26, 31, 34, 38, 43, 56, 57, 64, 66, 74, 75, 76 of SEQ ID NO: 1, preferably wherein the modification is a substitution.

In some embodiments, a ubiquitin masking domain is provided, wherein the first masking domain comprising a portion of a ubiquitin or ubiquitin variant and the second masking domain comprising a portion of a ubiquitin or ubiquitin variant comprise one or more interacting surfaces. In some embodiments, the first masking domain and/or the second masking domain comprise a portion of a ubiquitin variant, which comprises one or more substitutions at one or more interacting surfaces of the first masking domain and the second masking domain.

In some embodiments, a ubiquitin masking domain is provided, wherein the first masking domain comprising a portion of a ubiquitin or ubiquitin variant and the second masking domain comprising a portion of a ubiquitin or ubiquitin variant interact to form a globular structure.

In some embodiments, a ubiquitin masking domain comprising a ubiquitin variant is provided, wherein the ubiquitin variant comprises one or more substitutions at a position corresponding to position Q2; positions Q2 and E64; positions F4 and T66; positions K11 and E34; positions P19 and S57; positions Q31 and P38; position L56; and/or position T66 of SEQ ID NO: 1.

In some embodiments, a ubiquitin masking domain comprising a ubiquitin variant is provided, wherein the ubiquitin variant comprises one or more of, such as two or more of, three or more of, four or more of, or all five of the following pairs of substitutions: Q2C and E64C; F4C and T66C; K11C and E34C; P19C and S57C; and/or Q31C and P38C of SEQ ID NO: 1.

In some embodiments, a ubiquitin masking domain comprising a ubiquitin variant is provided, wherein the ubiquitin variant comprises a substitution Q2D/E/K/R; F4D/E/K/R; K11D/E/R; P19D/E/K/R; Q31D/E/K/R; E34D/K/R; P38D/E/K/R; S57D/E/K/R; and/or T66D/E/K/R of SEQ ID NO: 1.

In some embodiments, a ubiquitin masking domain comprising a ubiquitin variant is provided, wherein the variant comprises a substitution Q2K/R; a substitution E34D; a substitution L43F; a substitution L56K; a substitution T66K; and/or comprises a pair of substitutions F4D/E and T66K/R; K11R and E34D; and/or Q31R and P38D of SEQ ID NO: 1. In some embodiments, the variant comprises a substitution Q2D/E/K/R, such as Q2K of SEQ ID NO: 1.

In some embodiments, a ubiquitin masking domain comprising a ubiquitin variant is provided, wherein the N-terminal portion of the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 2, and wherein the variant comprises a modification at one or more positions corresponding to positions 2, 4, 5, 11, 19, 26, 31, of SEQ ID NO: 2, preferably wherein the modification is a substitution.

In some embodiments, a ubiquitin masking domain comprising a ubiquitin variant is provided, wherein the ubiquitin variant comprises a substitution V5L of SEQ ID NO: 1 or SEQ ID NO: 2.

In some embodiments, a ubiquitin masking domain comprising a ubiquitin variant is provided, wherein the ubiquitin variant comprises a substitution V26F/L of SEQ ID NO: 1 or SEQ ID NO: 2.

In some embodiments, a ubiquitin masking domain comprising a ubiquitin variant is provided, wherein the C-terminal portion of the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 3, and wherein the variant comprises a modification at one or more positions corresponding to positions 1, 4, 9, 40, 41, 42 SEQ ID NO: 3, preferably wherein the modification is a substitution.

In some embodiments, a ubiquitin masking domain comprising a ubiquitin variant is provided, wherein the ubiquitin variant comprises a substitution L9F of SEQ ID NO: 3.

In some embodiments, a ubiquitin masking domain comprising a ubiquitin variant is provided, wherein the ubiquitin variant comprises a substitution R40P of SEQ ID NO: 3.

In some embodiments, a ubiquitin masking domain comprising a ubiquitin variant is provided, wherein the C-terminus of SEQ ID NO: 1, SEQ ID NO: 2, and/or SEQ ID NO: 3 comprises a linker, e.g., of the form S(G)nS, wherein n is from 5-20; or of the form (G)n, wherein n is an integer of at least one, such as Gly-Gly; or of the form (GS)n, wherein n is an integer of at least one, such as Gly-Ser; such as a linker of SEQ ID NO:s 44-55, such as SEQ ID NO: 44 or SEQ ID NO: 50.

In some embodiments, a ubiquitin masking domain comprising a ubiquitin or ubiquitin variant is provided, wherein the N-terminal portion of the ubiquitin or ubiquitin variant has at least 90% sequence identity, such as at least 92%, at least 93%, at least 94%, at least 95%, at least 96%, at least 97%, at least 98%, at least 99% or 100% sequence identity to any one of SEQ ID NOs: 2, 6-10, 18-32, 66-94.

In some embodiments, a ubiquitin masking domain comprising a ubiquitin or ubiquitin variant is provided, wherein the N-terminal portion of the ubiquitin or ubiquitin variant comprises or consists of any one of SEQ ID NOs: 2, 6-10, 18-32, 66-94.

In some embodiments, a ubiquitin masking domain comprising a ubiquitin or ubiquitin variant is provided, wherein the C-terminal portion of the ubiquitin or ubiquitin variant has at least 90% sequence identity, such as at least 92%, at least 93%, at least 94%, at least 95%, at least 96%, at least 97%, at least 98%, at least 99% or 100% sequence identity to any one of SEQ ID NOs: 3-5, 11-17, 33-43, 66-94.

In some embodiments, a ubiquitin masking domain comprising a ubiquitin or ubiquitin variant is provided, wherein the C-terminal portion of the ubiquitin comprises or consists of any one of SEQ ID NOs: 3-5, 11-17, 33-43, 66-94.

In some embodiments, the first masking domain and the second masking domain comprise the sequences of SEQ ID NOs: 2 and 4, respectively; or SEQ ID NOs: 2 and 5, respectively; or SEQ ID NOs: 6 and 12, respectively; or SEQ ID NOs: 7 and 13, respectively; or SEQ ID NOs: 8 and 14, respectively; or SEQ ID NOs: 9 and 15, respectively, or SEQ ID NOs: 10 and 16, respectively, or SEQ ID NOs: 7 and 17, respectively; or SEQ ID NOs: 11 and 18, respectively; or SEQ ID NOs: 19 and 12, respectively; or SEQ ID NOs: 20 and 13, respectively; or SEQ ID NOs: 21 and 14, respectively; or SEQ ID NOs: 22 and 12, respectively; SEQ ID NOs: 23 and 34, respectively; or SEQ ID NOs: 24 and 35, respectively; or SEQ ID NOs: 25 and 36, respectively; or SEQ ID NOs: 26 and 36, respectively; or SEQ ID NOs: 25 and 38, respectively; or SEQ ID NOs: 26 and 38, respectively; or SEQ ID NOs: 27 and 37, respectively; or SEQ ID NOs: 28 and 39, respectively; or SEQ ID NOs: 29 and 40, respectively; or SEQ ID NOs: 29 and 41, respectively; or SEQ ID NOs: 30 and 40, respectively; or SEQ ID NOs: 30 and 41, respectively; or SEQ ID NOs: 31 and 13, respectively; or SEQ ID NOs: 32 and 13, respectively; or SEQ ID NOs: 6 and 33, respectively; or SEQ ID NOs: 6 and 42, respectively; or SEQ ID NOs: 7 and 43, respectively.

In any of the embodiments described herein, each masking domain may comprise a protease-cleavable linker. In some such embodiments, the masking domain is linked to the heavy chain or light chain via the protease-cleavable linker.

In some cases, antigen binding is reduced at least about 30-fold by the presence of a masking domain (e.g., a ubiquitin masking domain). In some embodiments, antigen binding is reduced by at least about 50-fold, such as at least about 80-fold, or at least about 100-fold by the presence of a masking domain (e.g., a ubiquitin masking domain).

In some embodiments, cytotoxicity of the conjugate is reduced at least 30-fold by the presence of a masking domain (e.g., a ubiquitin masking domain). In some embodiments, cytotoxicity of the conjugate is reduced at least 50-fold, such as at least about 80-fold, or at least about 100-fold by the presence of a masking domain (e.g., a ubiquitin masking domain).

In some embodiments, additional amino acids other than those described herein are substituted in the ubiquitin masking domain. In some such embodiments, the additional substitution(s) do not significantly alter the properties of the ubiquitin masking domain comprising the substitution. In certain exemplary embodiments, additional amino acid substitutions are conservative substitutions. For purposes of classifying amino acid substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine.

### Linkers and Cleavage Sites

In certain embodiments of the invention, a masking domain comprises a linker, which is located between the ubiquitin masking domain and the antibody chain to which the ubiquitin masking domain is attached. The linkers can be any segments of amino acids conventionally used as linkers for joining peptide domains. Suitable linkers can vary in length, such as from 1-20, 2-15, 3-12, 4-10, 5, 6, 7, 8, 9 or 10 amino acid. Some such linkers include a segment of polyglycine. Some such linkers include one or more serine residues, often at positions flanking the glycine residues. Other linkers include one or more alanine residues. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). Some exemplary linkers are in the form S(G)nS, wherein n is from 5-20. Other exemplary linkers are (G)n, such as Gly-Gly, glycine-serine polymers (including, for example, (GS)n, e.g, Gly-Ser, (GSGGS)n ((GSGGS) is SEQ ID NO: 44) and (GGGS)n ((GGGS) is SEQ ID NO: 45), where n is an integer of at least one), glycine-alanine polymers, alanine-serine polymers, and other flexible linkers known in the art. Some examples of linkers are Ser-(Gly)10-Ser (SEQ ID NO: 46), Gly-Gly-Ala-Ala (SEQ ID NO: 47), Gly-Gly-Gly-Gly-Ser (SEQ ID NO: 48), Leu-Ala-Ala-Ala-Ala (SEQ ID NO: 49), Gly-Gly-Ser-Gly (SEQ ID NO: 50), Gly-Gly-Ser-Gly-Gly (SEQ ID NO: 51), Gly-Ser-Gly-Ser-Gly (SEQ ID NO: 52), Gly-Ser-Gly-Gly-Gly (SEQ ID NO: 53), Gly-Gly-Gly-Ser-Gly (SEQ ID NO: 54), Gly-Ser-Ser-Ser-Gly (SEQ ID NO: 55), and the like.

The protease site is preferably recognized and cleaved by a protease expressed extracellularly so it contacts a masked antibody, releasing the masked antibody and allowing it to contact its target, such as a receptor extracellular domain or soluble ligand. Several matrix metalloproteinase sites (MMP1-28) are suitable. MMPs play a role in tissue remodeling and are implicated in neoplastic processes such as morphogenesis, angiogenesis and metastasis. Some exemplary protease sites are PLG-XXX (SEQ ID NO: 56), a well-known endogenous sequence for MMPs, PLG-VR (WO2014193973; SEQ ID NO: 61) and IPVSLRSG (SEQ ID NO: 57) (Turk et al., Nat. Biotechnol., 2001, 19, 661-667), LSGRSDNY (SEQ ID NO: 60) (Cytomx) and GPLGVR (SEQ ID NO: 59) (Chang et al., Clin. Cancer Res. 2012 Jan. 1; 18(1):238-47). Additional examples of MMPs are provided in US 2013/0309230, WO 2009/025846, WO 2010/081173, WO 2014/107599, WO 2015/048329, US 20160160263, and Ratnikov et al., Proc. Natl. Acad. Sci. USA, 111: E4148-E4155 (2014).

TABLE 1
Protease cleavage sequences. The MMP-cleavage
site is indicated by * while the
uPA/matriptase/legumain cleavage
sites are indicated by **.

| Cleavage Site Name | Sequence |
|---|---|
| M2 | GPLG*VR** (SEQ ID NO: 59) |
| IPV | IPVS*LR** SG (SEQ ID NO: 57) |

Other exemplary protease sites are PLGLAG (SEQ ID NO: 62), LALGPG (SEQ ID NO: 63), and YGRAA (SEQ ID NO: 64). In particular, the LALGPG (SEQ ID NO: 63) sequence is a scrambled version of PLGLAG (SEQ ID NO: 62) and is a protease-resistant sequence. See, e.g., Jiang, T., et al., Proc Natl Acad Sci USA. 2004 Dec. 21; 101(51): 17867-17872.

In various embodiments, a masking domain comprises a ubiquitin domain, a linker, and a protease cleavage sequence. In some such embodiments, the linker and the protease cleavage site comprise the sequence GSIPVSLRSG (SEQ ID NO: 58). In various embodiments, a masked antibody comprises two different masking domains (i.e., comprising different ubiquitin domains provided herein), each of which comprises the same protease site sequence, such as the linker-protease site sequence GSIPVSLRSG (SEQ ID NO: 58).

### II. Pharmaceutical Compositions

For therapeutic use, a masked antibody is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the compositions and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, masked antibodies of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

In some embodiments, compositions of masked antibodies are aqueous compositions. In other embodiments, the compositions are lyophilized.

Pharmaceutical compositions of a masked antibody as disclosed herein can be presented in a dosage unit form, or can be stored in a form suitable for supplying more than one unit dose. A pharmaceutical composition should be formulated to be compatible with its intended route of administration. Lyophilized formulations are typically reconstituted in solution prior to administration or use, whereas aqueous formulations may be “ready to use,” meaning that they are administered directly, without being first diluted for example, or can be diluted in saline or another solution prior to use.

Examples of routes of administration are intravenous (IV), intradermal, intratumoral, inhalation, transdermal, topical, transmucosal, and rectal administration. The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Pharmaceutical compositions are preferably sterile. Sterilization can be accomplished by any suitable method, e.g., filtration through sterile filtration membranes. Where the composition is lyophilized, filter sterilization can be conducted prior to or following lyophilization and reconstitution.

The present invention also provides a kit, comprising packaging material and at least one vial comprising a composition of masked antibody as described herein. The kit may further comprise instructions for use and/or a diluent solution if the antibody formulation must be diluted prior to use. The present invention also provides a kit, comprising packaging material and at least one vial comprising a lyophilized composition of masked antibody as described herein. The kit may further comprise instructions for use, a reconstitution solution for reconstituting the antibody into solution, and/or a diluent solution if the antibody composition is to be further diluted after reconstitution.

### II. Exemplary Antibodies

Antibodies include non-human, humanized, human, chimeric, and veneered antibodies, nanobodies, dAbs, scFV's, Fabs, and the like. Some such antibodies are immunospecific for a cancer cell antigen, preferably one on the cell surface internalizable within a cell on antibody binding. In some embodiments, the antibody portion of a masked antibody binds a therapeutic antigen. Such therapeutic antigens include antigens that may be targeted for treatment of any disease or disorder, including, but not limited to, cancer, autoimmune disorders, and infections.

Targets to which antibodies can be directed include receptors on cancer cells and their ligands or counter-receptors (i.e., tumor-associated antigens). Such targets include, but are not limited to, CD3, CD19, CD20, CD22, CD30, CD33, CD34, CD40, CD44, CD47, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Axl, Clec12A, CD352, FAP, CXCR3, and CD5.

In some embodiments, a masked antibody provided herein may be useful for treating an autoimmune disease. Nonlimiting antigens that may be bound by an antibody useful for treating an autoimmune disease include TNF-α, IL-1, IL-2R, IL-6, IL-12, IL-23, IL-17, IL-17R, BLyS, CD20, CD52, α4β7 integrin, and α4-integrin.

Some examples of commercial antibodies and their targets suitable for use in the masked antibodies described herein include, but are not limited to, brentuximab or brentuximab vedotin, CD30, alemtuzumab, CD52, rituximab, CD20, trastuzumab Her/neu, nimotuzumab, cetuximab, EGFR, bevacizumab, VEGF, palivizumab, RSV, abciximab, GpIIb/IIIa, infliximab, adalimumab, certolizumab, golimumab TNF-alpha, baciliximab, daclizumab, IL-2R, omalizumab, IgE, gemtuzumab or vadastuximab, CD33, natalizumab, VLA-4, vedolizumab alpha4beta7, belimumab, BAFF, otelixizumab, teplizumab CD3, ofatumumab, ocrelizumab CD20, epratuzumab CD22, alemtuzumumab CD52, eculizumab C5, canakimumab IL-1beta, mepolizumab IL-5, reslizumab, tocilizumab IL-6R, ustekinumab, briakinumab IL-12, 23, hBU12 (CD19) (US20120294853), humanized 1F6 or 2F12 (CD70) (US20120294863), BR2-14a and BR2-22a (LIV-1) (WO2012078688).

### Glycosylation Variants

Antibodies may be glycosylated at conserved positions in their constant regions (Jefferis and Lund, (1997) Chem. Immunol. 65:111-128; Wright and Morrison, (1997) TibTECH 15:26-32). The oligosaccharide side chains of the immunoglobulins affect the protein's function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Jefferis and Lund, supra; Wyss and Wagner, (1996) Current Op. Biotech. 7:409-416). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., (1995) Nature Med. 1:237-243). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., (1996) Mol. Immunol. 32:1311-1318), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of α(1,4)-N-acetylglucosaminyltransferase III (GnTIII), a glycosyltransferase catalyzing formation of bisecting GlcNAc, was reported to have improved ADCC activity (Umana et al. (1999) Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc.

Addition of glycosylation sites to an antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of the antibody.

The amino acid sequence is usually altered by altering the underlying nucleic acid sequence. These methods include isolation from a natural source (in the case of naturally-occurring amino acid sequence variants) or preparation by oligonucleotide-mediated (or site-directed) mutagenesis, PCR mutagenesis, and cassette mutagenesis of an earlier prepared variant or a non-variant version of the antibody.

The glycosylation (including glycosylation pattern) of antibodies may also be altered without altering the amino acid sequence or the underlying nucleotide sequence. Glycosylation largely depends on the host cell used to express the antibody. Since the cell type used for expression of recombinant glycoproteins, e.g., antibodies, as potential therapeutics is rarely the native cell, significant variations in the glycosylation pattern of the antibodies can be expected. See, e.g., Hse et al., (1997) J. Biol. Chem. 272:9062-9070. In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261; 5,278,299). Glycosylation, or certain types of glycosylation, can be enzymatically removed from the glycoprotein, for example using endoglycosidase H (Endo H). In addition, the recombinant host cell can be genetically engineered, e.g., make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

A preferred form of modification of glycosylation of antibodies is reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

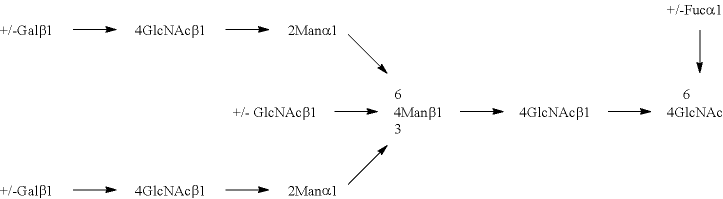

where +/− indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to certain methods, only a minor amount of fucose is incorporated into the complex N-glycoside-linked sugar chain(s) of an antibody. For example, in various embodiments, less than about 60%, less than about 50%, less than about 40%, less than about 30%, less than about 20%, less than about 15%, less than about 10%, less than about 5%, or less than about 3% of the molecules of an antibody have core fucosylation by fucose. In some embodiments, about 2% of the molecules of the antibody has core fucosylation by fucose.

In certain embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the antibodies have core fucosylation by a fucose analog or a metabolite or product of the fucose analog. In some embodiments, about 2% of the antibodies have core fucosylation by a fucose analog or a metabolite or product of the fucose analog.

Methods of making non-fucosylated antibodies (which may be used to make non-fucosylated masked antibodies) by incubating antibody-producing cells with a fucose analogue are described, e.g., in WO2009/135181. Briefly, cells that have been engineered to express the antibody are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In one embodiment, the fucose analogue is 2-flurofucose. Methods of using fucose analogues in growth medium and other fucose analogues are disclosed, e.g., in WO/2009/135181, which is herein incorporated by reference.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a Golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that would be able to produce a non-fucosylated antibody.

Many methods are available to determine the amount of fucosylation on an antibody. Methods include, e.g., LC-MS via PLRP-S chromatography and electrospray ionization quadrupole TOF MS.

### IV. Linking Ubiquitin Masking Agents to Antibodies

A portion of a ubiquitin protein is linked to the amino-termini of antibody variable regions via a linker including a protease site. A typical antibody includes a heavy and light chain variable region, in which case a portion of a ubiquitin protein is linked to the amino-termini of each. A bivalent antibody has two binding sites, which may or may not be the same. In a normal monospecific antibody, the binding sites are the same and the antibody has two identical light and heavy chain pairs. In this case, each heavy chain is linked to the same portion of a ubiquitin protein and each light chain to the same portion of a ubiquitin protein (which may or may not be the same as the portion of a ubiquitin protein linked to the heavy chain). In a bispecific antibody, the binding sites are different and formed from two different heavy and light chain pairs. In such a case, the heavy and light chain variable region of one binding site are respectively linked to portions of a ubiquitin protein as are the heavy and light chain variable regions of the other binding site. Typically, both heavy chain variable regions are linked to the same portion of a ubiquitin protein as are both light chain variable regions.

A portion of a ubiquitin protein can be linked to an antibody variable region via a linker including a protease site. Typically, the same linker with the same protease cleavage site is used for linking each heavy or light chain variable region of an antibody to a portion of a ubiquitin protein. The protease cleavage site should be one amenable to cleavage by a protease present extracellularly in the intended target tissue or pathology, such as a cancer, such that cleavage of the linker releases the antibody from the ubiquitin masking its activity allowing the antibody to bind to its intended target, such as a cell-surface antigen or soluble ligand.

As well as the variable regions, a masked antibody typically includes all or part of a constant region, which can include any or all of a light chain constant region, CH1, hinge, CH2 and CH3 regions. As with other antibodies one or more carboxy-terminal residues can be proteolytically processed or derivatized.

Each antibody chain can be linked to a single portion of a ubiquitin protein or multiple such peptides in tandem (e.g., two, three, four or five copies of the portion of a ubiquitin protein). If the latter, the peptides in tandem linkage are usually the same. Also, if tandem linkage is employed, light and heavy chains are usually linked to the same number of peptides.

Linkage of antibody chains to a portion of a ubiquitin protein can reduce the binding affinity of an antibody by at least about 10-fold, at least about 20-fold, at least about 30-fold, at least about 40-fold, at least about 50-fold, at least about 80-fold, or at least about 100-fold relative to the same antibody without such linkage or after cleavage of such linkage. In some such antibodies, binding affinity is reduced between about 10-100-fold, between about 30-100-fold, between about 50-100-fold, between about 80-100-fold, between about 10-50-fold, between about 20-50-fold, between about 30-50-fold, between about 40-50-fold, between about 30-80-fold, between about 40-80-fold, or between about 50-80-fold. Effector functions of the antibody, such as ADCC, phagocytosis, and CDC or cytotoxicity as a result of linkage to a drug in an antibody drug conjugate can be reduced by the same factors or ranges. Upon proteolytic cleavage that serves to unmask an antibody or otherwise remove the mask from the antibody, the restored antibody typically has an affinity or effect function that is within a factor of 2, 1.5 or preferably unchanged within experimental error compared with an otherwise identical control antibody, which has never been masked.

### V. Antibody-Drug Conjugates

In certain embodiments, a masked antibody may comprise an antibody drug conjugates (ADCs, also referred to herein as an “immunoconjugate”). Particular ADCs may comprise cytotoxic agents (e.g., chemotherapeutic agents), prodrug converting enzymes, radioactive isotopes or compounds, or toxins (these moieties being collectively referred to as a therapeutic agent). For example, an ADC can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA replication inhibitors, DNA alkylating agents, NAMPT inhibitors, and tubulin inhibitors (i.e., antitubulins). Exemplary cytotoxic agents include, for example, auristatins, camptothecins, calicheamicins, duocarmycins, etoposides, enediyine antibiotics, maytansinoids (e.g., DM1, DM2, DM3, DM4), taxanes, benzodiazepines (e.g., pyrrolo[1,4]benzodiazepines, indolinobenzodiazepines, and oxazolidinobenzodiazepines including pyrrolo[1,4]benzodiazepine dimers, indolinobenzodiazepine dimers, and oxazolidinobenzodiazepine dimers), lexitropsins, taxanes, combretastatins, cryptophysins, and vinca alkaloids. Nonlimiting exemplary cytotoxic agents include auristatin E, AFP, AEB, AEVB, MMAF, MMAE, auristatin T (AT), paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, and maytansine. In certain embodiments, the cytotoxic agent includes MMAF or MMAE.

An ADC can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14: 1-9; Senter, Cancer J., 2008, 14(3): 154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the antigen-expressing cancer cell but is not substantially sensitive to the extracellular environment, such that the conjugate is cleaved from the antibody when it is internalized by the antigen-expressing cancer cell (e.g., in the endosomal or, for example by virtue of pH sensitivity or protease sensitivity, in the lysosomal environment or in the caveolear environment). In some embodiments, the therapeutic agent can also be attached to the antibody with a non-cleavable linker.

In certain exemplary embodiments, an ADC can include a linker region between a cytotoxic or cytostatic agent and the antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in antigen-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

A cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al, Biol. Chem. 264: 14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al, Anticancer Res. 15: 1387-93, 1995), a maleimidobenzoyl linker (Lau et al., Bioorg-Med-Chem. 3: 1299-1304, 1995), or a 3′-N-amide analog (Lau et al., Bioorg-Med-Chem. 3: 1305-12, 1995).

The linker also can be a non-cleavable linker, such as an maleimido-alkylene or maleimide-aryl linker that is directly attached to the therapeutic agent and released by proteolytic degradation of the antibody.

Typically, the linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC is cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

The linker can also promote cellular internalization. The linker can promote cellular internalization when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antibody (i.e., in the milieu of the ADC as described herein).

The antibody can be conjugated to the linker via a heteroatom of the antibody. These heteroatoms can be present on the antibody in its natural state or can be introduced into the antibody. In some aspects, the antibody will be conjugated to the linker via a nitrogen atom of a lysine residue. In other aspects, the antibody will be conjugated to the linker via a sulfur atom of a cysteine residue. Methods of conjugating linker and drug-linkers to antibodies are known in the art.

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically, the auristatin based antibody-drug conjugate comprises a linker between the auristatin drug and the antibody. The linker can be, for example, a cleavable linker (e.g., a peptidyl linker) or a non-cleavable linker (e.g., linker released by degradation of the antibody). Auristatins include MMAF, and MMAE. The synthesis and structure of exemplary auristatins are described in U.S. Pat. Nos. 7,659,241, 7,498,298, 2009-0111756, 2009-0018086, and U.S. Pat. No. 7,968,687 each of which is incorporated herein by reference in its entirety and for all purposes.

Other exemplary antibody-drug conjugates include maytansinoid antibody-drug conjugates meaning that the drug component is a maytansinoid drug, and benzodiazepine antibody drug conjugates meaning that the drug component is a benzodiazepine (e.g., pyrrolo[1,4]benzodiazepine dimers, indolinobenzodiazepine dimers, and oxazolidinobenzodiazepine dimers).

In certain embodiments, an antibody may be combined with an ADC with binding specificity to a different target. Exemplary ADCs that may be combined with a masked antibody include brentuximab vedotin (anti-CD30 ADC), enfortumab vedotin (anti-nectin-4 ADC), ladiratuzumab vedotin (anti-LIV-1 ADC), denintuzumab mafodotin (anti-CD19 ADC), glembatumumab vedotin (anti-GPNMB ADC), anti-TIM-1 ADC, polatuzumab vedotin (anti-CD79b ADC), anti-MUC16 ADC, depatuxizumab mafodotin, telisotuzumab vedotin, anti-PSMA ADC, anti-C4.4a ADC, anti-BCMA ADC, anti-AXL ADC, tisotuumab vedotin (anti-tissue factor ADC).

### VI. Masked Antibody Expression

Nucleic acids encoding masked antibodies can be expressed in a host cell that contains endogenous DNA encoding a masked antibody used in the present invention. Such methods are well known in the art, e.g., as described in U.S. Pat. Nos. 5,580,734, 5,641,670, 5,733,746, and 5,733,761. Also see, e.g., Sambrook, et al., supra, and Ausubel, et al., supra. Those of ordinary skill in the art are knowledgeable in the numerous expression systems available for expression of a nucleic acid encoding a protein of the present invention. Illustrative of cell cultures useful for the production of the antibodies, masked antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, hep G2 cells, P3X63Ag8.653, SP2/0-Ag14, HeLa cells and the like, which are readily available from, for example, American Type Culture Collection, Manassas, VA. Yeast and bacterial host cells may also be used and are well known to those of skill in the art. Other cells useful for production of nucleic acids or proteins of the present invention are known and/or available, for instance, from the American Type Culture Collection Catalogue of Cell Lines and hybridomas or other known or commercial sources.

Expression vectors can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences). See, e.g., Ausubel et al., supra; Sambrook, et al., supra.

Expression vectors optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017), ampicillin, neomycin (G418), mycophenolic acid, or glutamine synthetase (GS, U.S. Pat. Nos. 5,122,464; 5,770,359; and 5,827,739), resistance for eukaryotic cell culture, and tetracycline or ampicillin resistance genes for culturing in E. coli and other bacteria or prokaryotes. Appropriate culture media and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra; Ausubel, supra.

The nucleic acid insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

The nucleic acid insert is optionally in frame with a portion of a ubiquitin protein sequence and/or an MMP cleavage sequence, e.g., at the amino-terminus of one or more heavy chain and/or light chain sequences. Alternatively, a portion of a ubiquitin protein sequence and/or an MMP cleavage sequence can be post-translationally added to an antibody, e.g., via a disulfide bond or the like.

When eukaryotic host cells are employed, polyadenylation or transcription terminator sequences are typically incorporated into the vector. An example of a terminator sequence is the polyadenylation sequence from the bovine growth hormone gene. Sequences for accurate splicing of the transcript can also be included. An example of a splicing sequence is the VP1 intron from SV40 (Sprague, et al. (1983) J. Virol. 45:773-781). Additionally, gene sequences to control replication in the host cell can be incorporated into the vector, as known in the art.

### VI. Masked Antibody Isolation and Purification

Masked antibodies used in the present formulations can be recovered and purified from recombinant cell cultures by methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (HPLC) can also be employed for purification. See, e.g., Colligan, Current Protocols in Immunology, or Current Protocols in Protein Science, John Wiley & Sons, New York, N.Y., (1997-2001).

In some embodiments, antibodies or masked antibodies described herein can be expressed in a modified form. For instance, a region of additional amino acids, particularly charged amino acids, can be added to the amino-terminus of an antibody to improve stability and persistence in the host cell, during purification, or during subsequent handling and storage. Also, peptide moieties can be added to an antibody or masked antibody to facilitate purification. Such regions can be removed prior to final preparation of an antibody or masked antibody. Such methods are described in many standard laboratory manuals, such as Sambrook, supra; Ausubel, et al., ed., Current Protocols In Molecular Biology, John Wiley & Sons, Inc., NY, N.Y. (1987-2001).

Antibodies and masked antibodies described herein can include purified products, products of chemical synthetic procedures, and products produced by recombinant techniques from a eukaryotic host, including, for example, yeast, higher plant, insect and mammalian cells. Depending upon the host employed in a recombinant production procedure, the antibody or masked antibody of the present invention can be glycosylated or can be non-glycosylated, with glycosylated preferred. Such methods are described in many standard laboratory manuals, such as Sambrook, supra; Ausubel, supra, Colligan, Protein Science, supra.

### VIII. Therapeutic Applications

In some embodiments, masked antibodies herein may be used in methods of therapeutic treatment. Nonlimiting exemplary diseases and disorders that may be treated with the masked antibodies provided herein include cancer, autoimmune disorders, and infections. Generally, any disease or disorder that may be treated with a therapeutic antibody may be treated with a masked antibody provided herein. In some embodiments, a masked antibody results in reduced side-effects compared to the unmasked version of the antibody, for example, because the masked antibody does not bind its antigen until the mask has been removed. By selecting suitable cleavable linkers between the ubiquitin masking domains and the antibody chains, the masked antibody will remain masked until it reaches the vicinity of its target antigen, particularly its target antigen at the site of the disease or disorder. For example, in some instances, by selecting a cleavable linker that is cleaved by a protease that is present at higher concentration near a tumor, the masked antibody may have an improved safety profile because the antibody does not significantly bind its antigen until it reaches the tumor.

In some embodiments, methods of treating cancer are provided.

Positive therapeutic effects in cancer can be measured in a number of ways (See, W. A. Weber, J. Null. Med. 50:1S-10S (2009); Eisenhauer et al., supra). In some embodiments, response to a masked antibody is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a primary or a secondary hepatic cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Tumor” as it applies to a subject diagnosed with, or suspected of having, a primary or a secondary hepatic cancer, refers to a malignant or potentially malignant neoplasm or tissue mass of any size. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms). Nonlimiting exemplary sarcomas include soft tissue sarcoma and osteosarcoma.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

Nonlimiting exemplary autoimmune diseases that may be treated with a masked antibody include Crohn's disease, ulcerative colitis, rheumatoid arthritis, psoriatic arthritis, ankylosing spondylitis, uveitis, juvenile idiopathic arthritis, multiple sclerosis, psoriasis (including plaque psoriasis), systemic lupus erythematosus, granulomatosis with polyangiitis, microscopic polyangiitis, systemic sclerosis, idiopathic thrombocytopenic purpura, graft-versus-host disease, and autoimmune cytopenias.

As used herein, the term “effective amount” refers to the amount of a compound (e.g., a masked antibody) sufficient to effect beneficial or desired results. An effective amount can be administered in one or more administrations, applications or dosages and is not intended to be limited to a particular formulation or administration route. Generally, a therapeutically effective amount of active component is in the range of 0.01 mg/kg to 100 mg/kg, 0.1 mg/kg to 100 mg/kg, 1 mg/kg to 100 mg/kg, 0.01 mg/kg to 10 mg/kg, 0.1 mg/kg to 10 mg/kg, 1 mg/kg to 10 mg/kg. The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks.

In certain exemplary embodiments, the present invention provides a method for treating cancer in a cell, tissue, organ, animal or patient. In particular embodiments, the present invention provides a method for treating a solid cancer in a human. Examples of cancers include, but are not limited to, solid tumors, soft tissue tumors, hematopoietic tumors that give rise to solid tumors, and metastatic lesions. Examples of hematopoietic tumors that have the potential to give rise to solid tumors include, but are not limited to, diffuse large B-cell lymphomas (DLBCL), follicular lymphoma, myelodysplastic syndrome (MDS), a lymphoma, Hodgkin's disease, a malignant lymphoma, non-Hodgkin's lymphoma, Burkitt's lymphoma, multiple myeloma, Richter's Syndrome (Richter's Transformation) and the like. Examples of solid tumors include, but are not limited to, malignancies, e.g., sarcomas (including soft tissue sarcoma and osteosarcoma), adenocarcinomas, and carcinomas, of the various organ systems, such as those affecting head and neck (including pharynx), thyroid, lung (small cell or non-small cell lung carcinoma (NSCLC)), breast, lymphoid, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), central nervous system (e.g., neural or glial cells, e.g., neuroblastoma or glioma), skin (e.g., melanoma) and the like. In certain embodiments, the solid tumor is an NMDA receptor positive teratoma. In other embodiments, the cancer is selected from breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer.

In certain embodiments, the cancer is selected from, but not limited to, leukemias such as acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMoL).

In one embodiment, the cancer is a solid tumor that is associated with ascites. Ascites is a symptom of many types of cancer and can also be caused by a number of conditions, such as advanced liver disease. The types of cancer that are likely to cause ascites include, but are not limited to, cancer of the breast, lung, large bowel (colon), stomach, pancreas, ovary, uterus (endometrium), peritoneum and the like. In some embodiments, the solid tumor associated with ascites is selected from breast cancer, colon cancer, pancreatic cancer, stomach, uterine cancer, and ovarian cancer. In some embodiments, the cancer is associated with pleural effusions, e.g., lung cancer.

Additional hematological cancers that give rise to solid tumors include, but are not limited to, non-Hodgkin lymphoma (e.g., diffuse large B cell lymphoma, mantle cell lymphoma, B lymphoblastic lymphoma, peripheral T cell lymphoma and Burkitt's lymphoma), B-lymphoblastic lymphoma; B-cell chronic lymphocytic leukemia/small lymphocytic lymphoma; lymphoplasmacytic lymphoma; splenic marginal zone B-cell lymphoma (±villous lymphocytes); plasma cell myeloma/plasmacytoma; extranodal marginal zone B-cell lymphoma of the MALT type; nodal marginal zone B-cell lymphoma (±monocytoid B cells); follicular lymphoma; diffuse large B-cell lymphomas; Burkitt's lymphoma; precursor T-lymphoblastic lymphoma; T adult T-cell lymphoma (HTLV 1-positive); extranodal NK/T-cell lymphoma, nasal type; enteropathy-type T-cell lymphoma; hepatosplenic γ-δ T-cell lymphoma; subcutaneous panniculitis-like T-cell lymphoma; mycosis fungoides/sezary syndrome; anaplastic large cell lymphoma, T/null cell, primary cutaneous type; anaplastic large cell lymphoma, T-/null-cell, primary systemic type; peripheral T-cell lymphoma, not otherwise characterized; angioimmunoblastic T-cell lymphoma, multiple myeloma, polycythemia vera or myelofibrosis, cutaneous T-cell lymphoma, small lymphocytic lymphoma (SLL), marginal zone lymphoma, CNS lymphoma, immunoblastic large cell lymphoma, precursor B-lymphoblastic lymphoma and the like.

Masked antibodies as described herein can also be used to treat disorders associated with cancer, e.g., cancer-induced encephalopathy.

The masked antibodies can be used in methods of treatment in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., a synergistic response). The delivery can be such that an effect of the first treatment delivered is still detectable when the second is delivered.

In one embodiment, the methods of the invention include administering to the subject a masked antibody as described herein, e.g., in combination with one or more additional therapies, e.g., surgery or administration of another therapeutic preparation. In one embodiment, in the case of cancer, for example, the additional therapy may include chemotherapy, e.g., a cytotoxic agent. In one embodiment the additional therapy may include a targeted therapy, e.g. a tyrosine kinase inhibitor, a proteasome inhibitor, or a protease inhibitor. In one embodiment, the additional therapy may include an anti-inflammatory, anti-angiogenic, anti-fibrotic, or anti-proliferative compound, e.g., a steroid, a biologic immunomodulatory, such as an inhibitor of an immune checkpoint molecule, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In another embodiment, the additional therapy could include combining therapeutics of different classes. The antibody or masked antibody preparation and the additional therapy can be administered simultaneously or sequentially.

An “immune checkpoint molecule,” as used herein, refers to a molecule in the immune system that either turns up a signal (a stimulatory molecule) or turns down a signal (an inhibitory molecule). Many cancers evade the immune system by inhibiting T cell signaling. Hence, these molecules may be used in cancer treatments as additional therapeutics. In other cases, a masked antibody may be an immune checkpoint molecule.

Exemplary immune checkpoint molecules include, but are not limited to, programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, TGFR, adenosine 2A receptor (A2AR), B7-H3 (also known as CD276), B7-H4 (also called VTCN1), indoleamine 2,3-dioxygenase (IDO), 2B4, killer cell immunoglobulin-like receptor (KIR), and the like.

An “immune checkpoint inhibitor,” as used herein, refers to a molecule (e.g., a small molecule, a monoclonal antibody, an antibody fragment, etc.) that inhibit and/or block one or more inhibitory checkpoint molecules.

Exemplary immune checkpoint inhibitors include, but are not limited to, the following monoclonal antibodies: PD-1 inhibitors such as pembrolizumab (Keytruda, Merck) and nivolumab (Opdivo, Bristol-Myers Squibb); PD-L1 inhibitors such as atezolizumab (Tecentriq, Genentech), avelumab (Bavencio, Pfizer), durvalumab (Imfinzi, AstraZeneca); and CTLA-1 inhibitors such as ipilimumab (Yervoy, Bristol-Myers Squibb).

Exemplary cytotoxic agents include anti-microtubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a preparation described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

Assessment of proteases in tissues can be monitored using a variety of techniques, including both those that monitor protease activity as well as those that can detect proteolytic activity. Conventional methods that can detect the presence of proteases in a tissue, which could include both inactive and active forms of the protease, include IHC, RNA sequencing, Western blot, or ELISA-based methods. Additional techniques can be used to detect protease activity in tissues, which includes zymography, in situ zymography by fluorescence microscopy, or the use of fluorescent proteolytic substrates. In addition, the use of fluorescent proteolytic substrates can be combined with immuno-capture of specific proteases. Additionally, antibodies directed against the active site of a protease can be used by a variety of techniques including IHC, fluorescence microscopy, Western blotting, ELISA, or flow cytometry (See, Sela-Passwell et al. Nature Medicine. 18:143-147. 2012; LeBeau et al. Cancer Research. 75:1225-1235. 2015; Sun et al. Biochemistry. 42:892-900. 2003; Shiryaev et al. 2:e80. 2013.)

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Materials and Methods

All chemicals were purchased from Sigma Aldrich unless otherwise noted. Cancer cell lines were obtained from the American Type Culture Collection (ATCC). Cell culture media and media components were purchased from Gibco unless otherwise stated. MDpr-glucuronide-MMAE-maleimide and MDpr-glucuronide-AT-maleimide were prepared as previously described. (Lyon, R. P. et al. Nat. Biotechnol. 33, 7, 733-736, 2015).

### Example 1: Development of Masked Antibodies

Heterodimeric constructs with minimal aggregation and low potential for immunogenicity were assessed as antibody masks. To address these needs, a split protein system derived from ubiquitin (split Ubiquitin or “sUb”) was assessed. Ubiquitin is a small protein that is found in almost all tissues in eukaryotic organisms and serves as a post-translational modification. While ubiquitin is usually found intracellularly, it is also present in human plasma and serum at nanomolar concentrations. Split ubiquitin has been used as a sensor for protein-protein interactions which implies that the protein can maintain its globular structure despite having two ends appended to different proteins. Coupled with a low probability to cause an immune response, sUb can provide an antibody masking strategy.

### Example 2: Antibody Production

Antibodies were expressed via transient transfection of either human embryonic kidney (HEK) or Chinese hamster ovary (CHO) cells and purified using MabSelect SuRe Protein A resin (GE Healthcare Life Sciences). Further purification using size-exclusion chromatography (SEC) was performed for some antibodies, using a Superdex S200 column (GE Healthcare Life Sciences).

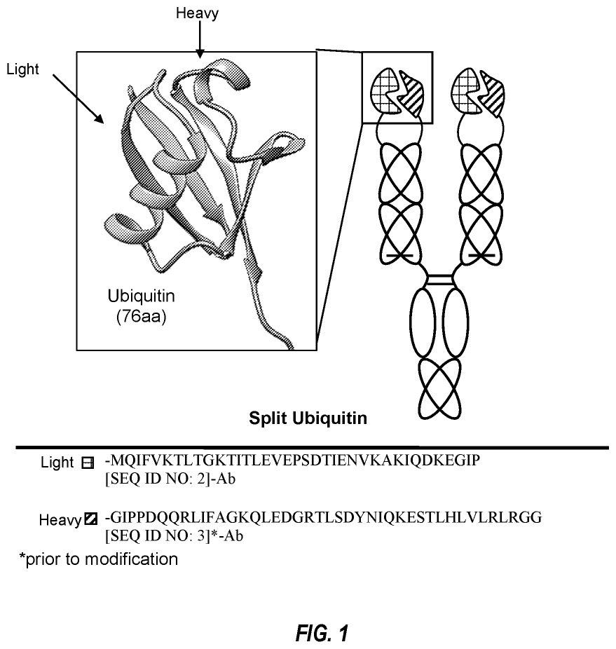

Using standard cloning techniques, the first 37 amino acids of ubiquitin, MQIFVKTLTGKTITLEVEPSDTIENVKAKIQDKEGIP (SEQ ID NO: 2) were fused to the N-terminus of the light chain, and the last 42 amino acids of ubiquitin, GIPPDQQRLIFAGKQLEDGRTLSDYNIQKESTLHLVLRLRGG (SEQ ID NO: 3) (FIG. 1), were fused to the N-terminus of the heavy chain. Positions 35-37 (PIG) of ubiquitin (SEQ ID NO: 1) are repeated in both the heavy and light chain to provide overlap.

Alternatively, the heavy chain of the antibody can contain the N-terminus of ubiquitin, and the light chain can contain the C-terminus of ubiquitin.

### Example 3: Aggregation Assessment

Antibody at 10 mg/mL in Phosphate Buffered Saline (PBS) was incubated at room temperature and at days 1, 2, 3, 4, 7, 9, 14, and 37, 2 μl of sample is diluted into 20 μl of PBS and assessed for aggregation using size exclusion chromatography.

### A. Size Exclusion Chromatography

Size exclusion chromatography (SEC) data were acquired on a Waters Acquity UPLC instrument. Samples were injected and separated on an analytical size exclusion column (Acquity UPLC Protein BEH SEC Column, 200A, 1.7 μm, 4.6×150 mm, part #186005225) in SEC Buffer (92.5% (25 mM sodium phosphate, 350 mM NaCl, pH 6.8), 7.5% IPA) at a flow rate of 0.4 mL/min for 6 minutes at RT.

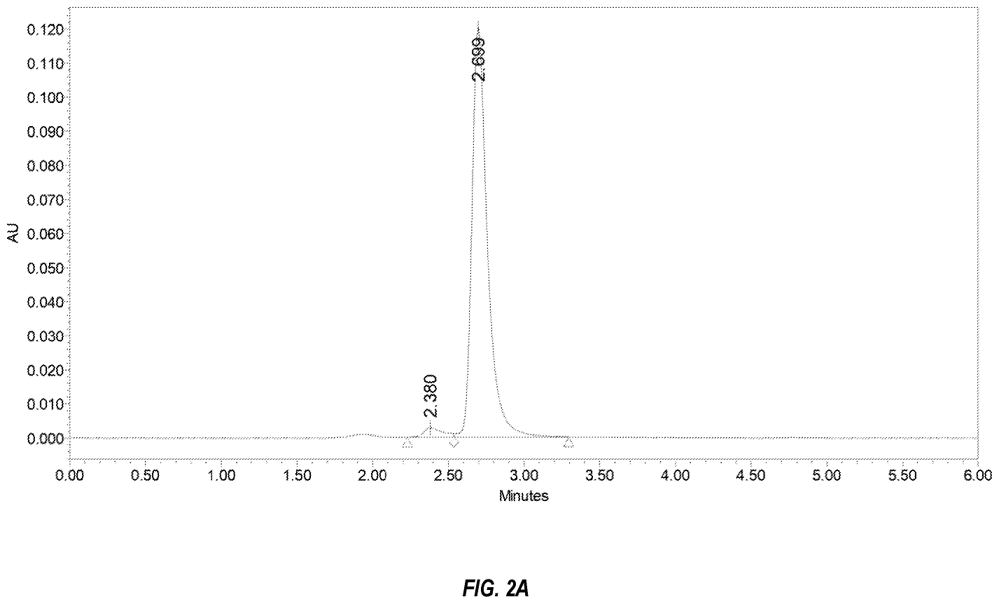

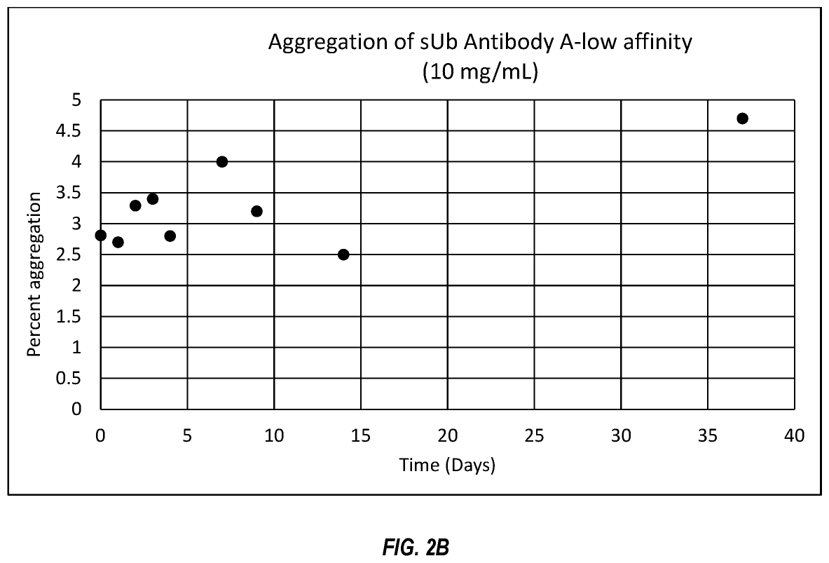

The antibody expressed with normal titers (˜100 mg/L) and were not aggregated upon purification (FIG. 2A). In a time course assessment, the percentage of aggregation stayed steady within the first 14 days and overall increased by 2% after 37 days at room temperature (FIG. 2B). These data show the sUb-masked antibodies remain stable at 10 mg/mL concentration at room temperatures for an extended period of time.

### Example 4: Masked Antibody Proteolysis

Matrix-metalloproteinase-2 (MMP-2) were activated via incubation with 1.25 mM 4-aminophenyl mercuric acetate (APMA) at 37° C. for 1 hour.

For all cleaved antibodies used in binding or cytotoxicity assays, masked antibody (ca. 50 μg) was incubated at 37° C. for 2-16 hours with activated human MMP-2 (100 U). The antibody was then purified using MabSelect SuRe Protein A resin (GE Healthcare Life Sciences). Briefly, antibody was bound to the resin at RT for at least 2 hours with constant mixing. The resin was then washed with PBS (5 column volumes) and antibody was eluted with 50 mM glycine pH 3.0. The eluent was then neutralized to pH 7.4 and buffer exchanged into PBS using dilution and concentration with Amicon 30 kDa molecular weight cutoff filters.

### Example 5: Modified C-Termini in Split Ubiquitin Mask

As a post-translational modification, ubiquitin is recognized by many deubiquitinases (DUBs) through the RGG motif found in the C-terminus of the protein. Two heavy chain C-termini were tested for the ability to mitigate the deubiquitination of the mask. The light chain was not further modified in this Example and, in each instance, comprised SEQ ID NO: 2. sUb PGG and sUb-Antibody A-low affinity antibodies were expressed in Expi293 cells and purified using protein A. The masses of the antibodies were assessed using quadrupole time of flight liquid chromatography-mass spectrometry (QTOF-LCMS).

### A. Mass Spectrometric Analysis of Antibodies or ADCs

Reverse-phase liquid chromatography-mass spectrometry (LC-MS) data were acquired on a Waters Xevo GS-S QTOF coupled to a Waters Acquity H-Class UPLC system. Samples were reduced with 10 mM dithiothreitol (DTT) for 10 min at 37° C. and then separated over an analytical reversed-phase column (Agilent Technologies, PLRP-S, 300 A, 2.1 mm ID×50 mm, 3 m) at 80° C. Samples were eluted using a linear gradient of 0.01% TFA in acetonitrile from 25% to 65% in 0.05% aqueous TFA over 5 minutes, followed by isocratic 65% 0.01% TFA in acetonitrile for 0.5 min at a flow rate of 1.0 mL/min. Mass spectrometry data was acquired in ESI+ mode using a mass range of 500-4000 m/z and were deconvoluted using MaxEnt1 to determine masses of the resulting antibodies or conjugates.

C-terminal modified heavy chain sequences are as in Table 2.

TABLE 2
C-terminal Modified Heavy Chain Sequences

| sUb | | Cleavage | Heavy Chain Mask (including linker | SEQ |
|---|---|---|---|---|
| Variant | Linker | Sequence | and cleavage sequence) | ID NO: |
| sUb-HC- | GS | n/a | GIPPDQQRLIFAGKQLEDGRTLSDYNIQKESTLHLV | 4 |
| PGG | | | LRLPGGGS | |
| sUb-HC- | GSGGS | n/a | GIPPDQQRLIFAGKQLEDGRTLSDYNIQKESTLHLV | 5 |
| GS | (SEQ ID NO: 44) | | LRLGSGGS | |

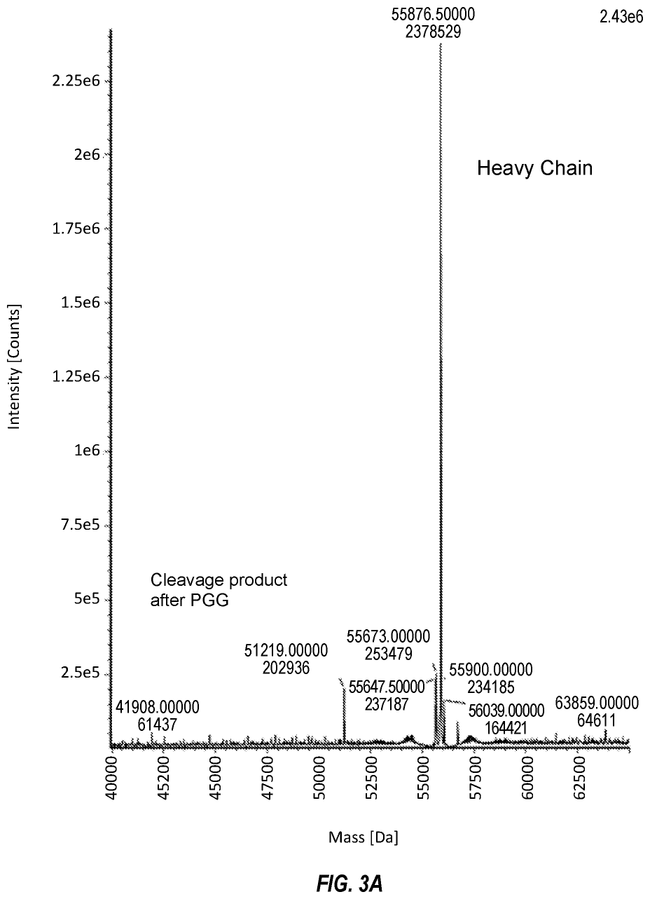

First, the C-terminal RGG of sUb was modified from RGG of the wild-type to PGG, to provide a masked antibody having heavy chain mask sUb-HC-PGG. This modification resulted in premature cleavage of the mask on the heavy chain during expression (FIG. 3A).

Second, the C-terminus was replaced with a “GS” linker to provide a masked antibody having heavy chain sUb-HC-GS. This masked antibody successfully mitigated the cleavage event (FIG. 3B).

### Example 6: Binding Ability of sUb-Masked Antibodies

A. Fluorescent Labeling of Antibodies Antibodies were fluorescently labeled using NHS ester activated Alexa Fluor 647 (Life Technologies) following the vendor protocol. Briefly, antibody (1 molar equivalent) was incubated with fluorophore (4-6 mol. equiv.) at pH 8 at room temperature (RT) for 1 hour. Fluorescent antibody was purified using Nap5 column (GE Healthcare Life Sciences) and fluorophore loading was quantified using UV-visible spectrophotometer (Agilent). Typical fluorophore loadings were 2-4 fluorophores per antibody.

### B. Competition Binding Experiments

To evaluate cell binding of masked antibodies, 2×105 Ramos cells were mixed with fluorescently-labeled parent antibody (0.6 nM) mixed with serial dilutions of competitor (masked antibody) in staining buffer (PBS, 2% FBS, 0.2% NaN3). Samples were incubated on ice for 1 hour and washed twice with ice-cold staining buffer. Labeled cells were examined by flow cytometry on a BD LSRII gated to exclude nonviable cells and analyzed using FlowJo Software. The IC50 was calculated using GraphPad Prism 6.

### C. Saturation Binding Experiments-Fluorescence-Activated Cell Sorting (FACs)

Binding of antibody or ADC to cell-surface antigen was assessed by flow cytometry on CD19+ Ramos, CD47+L540cy, CD47+SW780, αVβ6+ HEK cells. Cells (2×105) were combined with a serial dilution of indicated antibody in staining buffer (PBS, 2% FBS, 0.2% NaN3) in a total volume of 100 μL. The cells were incubated on ice for 1 hour and washed twice with ice-cold staining buffer. Cells were resuspended with anti-human IgG-AF647 (Jackson ImmunoResearch, 200-fold dilution in staining buffer) on ice for 1 hour. Cells were washed twice with ice cold staining buffer and resuspended in staining buffer. Labeled cells were examined by flow cytometry on an Attune N×T cytometer (Life Technologies) gated to exclude nonviable cells and analyzed using FlowJo Software. The EC₅₀ was calculated using nonlinear regression analysis in GraphPad Prism 6.

### D. Saturation Binding Experiments

ELISA plate (Nunc MaxiSorp™ flat-bottom, Thermo Fisher, 44-2404-21) wells were coated with 100 μl recombinant human integrin alpha V beta 6 protein (R&D, 3817AV050, 1 g/ml) or CD47 protein (Sino Biological, 12283-HCCH) overnight at 4° C. Protein in wells were combined with serial dilutions of indicated antibody in ELISA buffer (Tris Buffer Saline (TBS), 0.5% Tween (Fisher, AAJ60448AP), 1% BSA (A5503), 1 mM of MnCl2 (Sigma Aldrich M1787) and allowed to incubate for 1 hour at RT. Wells were washed 3× with TBS buffer with 0.5% Tween. Peroxidase AffiniPure F(ab′)₂ Fragment Goat Anti-Human IgG, Fcγ fragment specific (Jackson ImmunoResearch, 10,000-fold dilution in ELISA buffer, 109-036-098) was added to each well used for detecting testing antibody that are bound to the plate. Wells were washed 3× with TBS buffer with 0.5% Tween. To develop the HRP signal, 100 of TMB Chromogen Solution (Thermo Fisher, 00-202-3) was added to each well and fixed 100 μl of sulfonic acid (Sigma Aldrich). Signal was assessed using a SpectraMAX-190 (Molecular Devices) using reading at 450 nm and 630 nm.

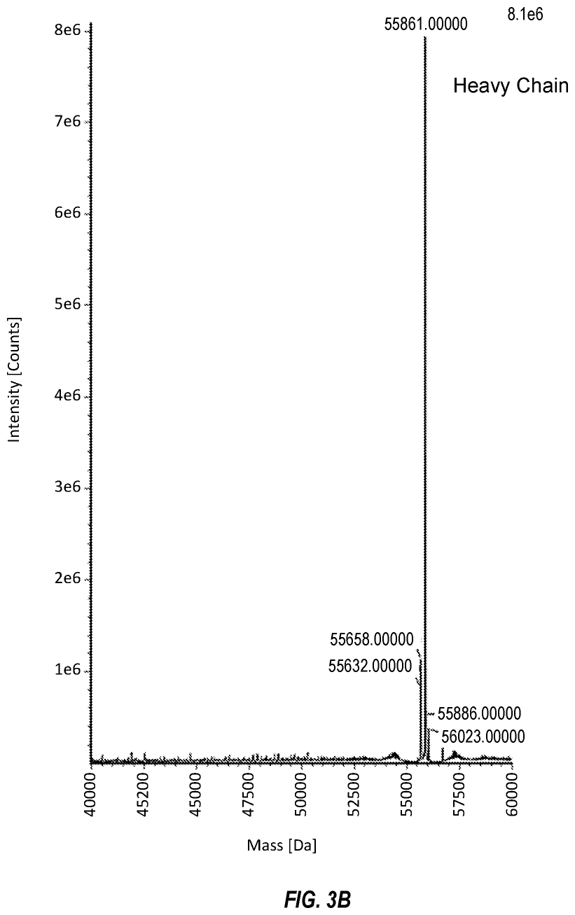

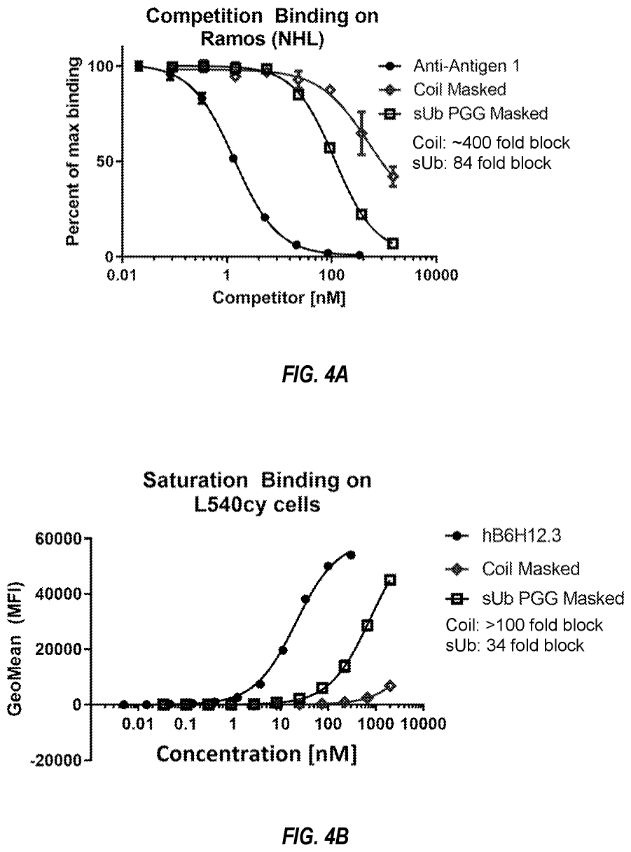

Briefly, coiled-coil masks (as described in, e.g., Trang et al., Nature Biotech. 37, 761-765 (2019)) and sUb-masked antibody having heavy chain mask sUb-HC-PGG (SEQ ID NO: 4), with light chain having mask of SEQ ID NO: 2, were applied to an antibody against Antigen-1 and hB6H12.3 against CD47 (FIG. 4A-4B). For Anti-Antigen-1, binding was assessed on Antigen-1-expressing Ramos cells by flow cytometry via competition with AlexaFluor 647-labeled Anti-Antigen-1. 50% of max binding of the parent antibody is 1.4 nM whereas the 50% of max binding for the coiled-coil-masked antibody is 1060 nM and for the sUb-masked antibody is 118 nM (FIG. 4A). For hB6H12.3, affinity was determined using saturation binding on CD47-expressing L540cy cells where the EC₅₀ of the parent antibody is 21 nM and EC₅₀ of the sUb-masked antibody is ˜720 nM and the affinity of the coiled-coil-masked antibody could not be determined because the experiment did not reach saturation (FIG. 4B). In both cases, the coiled-coil-masked antibody was able to reduce binding by over 100-fold whereas the sUb-masked antibody reduced binding by 34-84 fold. Due to the moderate blocking ability of the sUb-masked antibody, the sUb-mask will be applied to low affinity antibodies to further minimize the absolute affinity of the antibody.

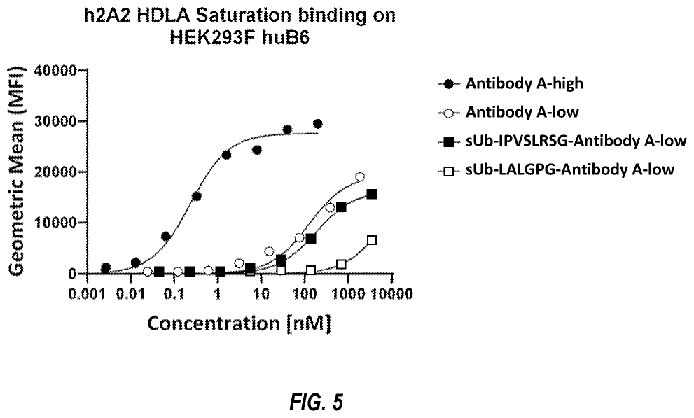

Using a low affinity αvβ6 targeting antibody, Antibody A, the effect of the linker sequence (IPVSLRSG (SEQ ID NO: 57) vs LALGPG (SEQ ID NO: 63)) on the blocking ability of sUb-masked Antibody A antibodies on HEK293F cells expressing human 36 was assessed (FIG. 5). The heavy chain and light chain were masked with the sequences shown in Table 3 (light chain) and in Table 4 (heavy chain), which include various linker sequences.

TABLE 3
Light chain masks

| sUb | | SEQ | Cleavage | Light Chain Mask (including | SEQ |
|---|---|---|---|---|---|
| Variant | Linker | ID NO: | Sequence | linker and cleavage sequence) | ID NO: |
| sUb-LC- | GGGS | 45 | IPV | MQIFVKTLTGKTITLEVEPSDTIENVKAKIQD | 6 |
| IPV | | | | KEGIPGGGSIPVSLRSG | |
| sUb-LC- | GGSG | 50 | PLG | MQIFVKTLTGKTITLEVEPSDTIENVKAKIQD | 7 |
| PLG | | | | KEGIPGGSGPLGLAGGS | |
| sUb-LC- | GGSG | 50 | Scr | MQIFVKTLTGKTITLEVEPSDTIENVKAKIQD | 8 |
| Scr | | | | KEGIPGGSGLALGPGGS | |
| SUbext- | GGGS | 45 | IPV | MQIFVKTLTGKTITLEVEPSDTIENVKAKIQD | 9 |
| LC-PGG | | | | KEGIPPDQQRLGGGS | |
| sUbext- | GGGS | 45 | IPV | MQIFVKTLTGKTITLEVEPSDTIENVKAKIQD | 10 |
| LC-IPV | | | | KEGIPPDQQRLGGGSIPVSLRSG | |
| sUb-LC- | GG | | IPV | GIPPDQQRLIFAGKQLEDGRTLSDYNIQKEST | 11 |
| flip | | | | LHLVLRLPGGGGIPVSLRSG | |

TABLE 4
Heavy chain masks

| sUb | | SEQ | Cleavage | Heavy Chain Mask (including | SEQ |
|---|---|---|---|---|---|
| Variant | Linker | ID NO: | Sequence | linker and cleavage sequence) | ID NO: |
| sUb-HC- | GSGGS | 44 | IPV | GIPPDQQRLIFAGKQLEDGRTLSDYNI | 12 |
| IPV | | | | QKESTLHLVLRLGSGGSIPVSLRSG | |
| sUb-HC- | GGSG | 50 | PLG | GIPPDQQRLIFAGKQLEDGRTLSDYNI | 13 |
| PLG | | | | QKESTLHLVLRLGGSGPLGLAGGS | |
| sUb-HC- | GGSG | 50 | Scr | GIPPDQQRLIFAGKQLEDGRTLSDYNI | 14 |
| Scr | | | | QKESTLHLVLRLGGSGLALGPGGS | |
| SUbext- | GS | | IPV | GIPPDQQRLIFAGKQLEDGRTLSDYNI | 15 |
| HC-PGG | | | | QKESTLHLVLRLPGGGS | |
| SUbext- | GS | | IPV | GIPPDQQRLIFAGKQLEDGRTLSDYNI | 16 |
| HC-IPV | | | | QKESTLHLVLRLPGGGSIPVSLRSG | |
| sUb-HC- | GGSG | 50 | PLG | GIPPDQQRLIFAGKQLEDGRTLSDYNI | 17 |
| short | | | | QKESTLHLVLGGSGPLGLAGGS | |
| sUb-HC- | GG | | IPV | MQIFVKTLTGKTITLEVEPSDTIENVK | 18 |
| flip | | | | AKIQDKEGIPGGGGIPVSLRSG | |

Masked antibody having the LALGPG (SEQ ID NO: 63) linker in each of the light chain and heavy chain (i.e., having, respectively, variants sUb-LC-Scr and sUb-HC-Scr) had minimal binding at concentrations up to 3.5 μM whereas masked-antibody having the IPVSLRSG linker on each of the light chain and heavy chains (i.e., having, respectively, variants sUb-LC-IPV and sUb-HC-IPV) had a EC₅₀ of ˜184 nM. These results indicate that changing the linker sequence can improve the blocking ability of the sUb-mask.

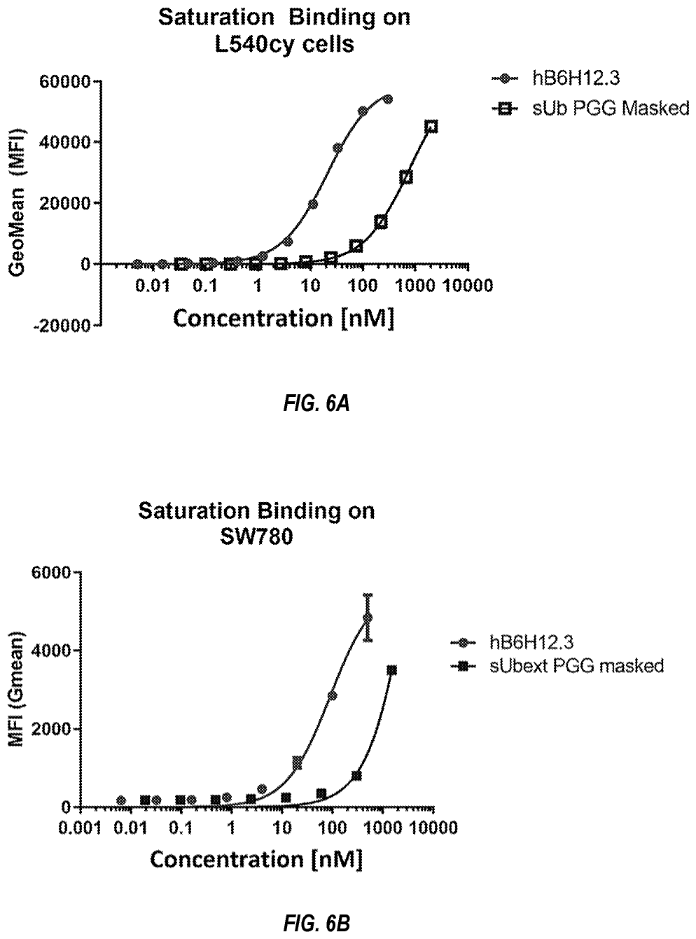

The effect of extending the overlap between the two ubiquitin portions on the heavy and light chain was also assessed. Affinity was assessed using saturation binding via flow cytometry on CD47-expressing cell lines. The-masked antibody having heavy chain mask sUb-HC-PGG (SEQ ID NO: 4) (and light chain having mask of SEQ ID NO: 2) reduced binding by 34-fold compared to the unmasked antibody (hB6H12.3) whereas the sUb-masked antibody having heavy chain sUbext-HC-PGG and light chain sUbext-LC-PGG reduced binding by 12-fold compared to the unmasked antibody. (FIG. 6A-6B).

### Example 7: Point Mutations in sUb Mask

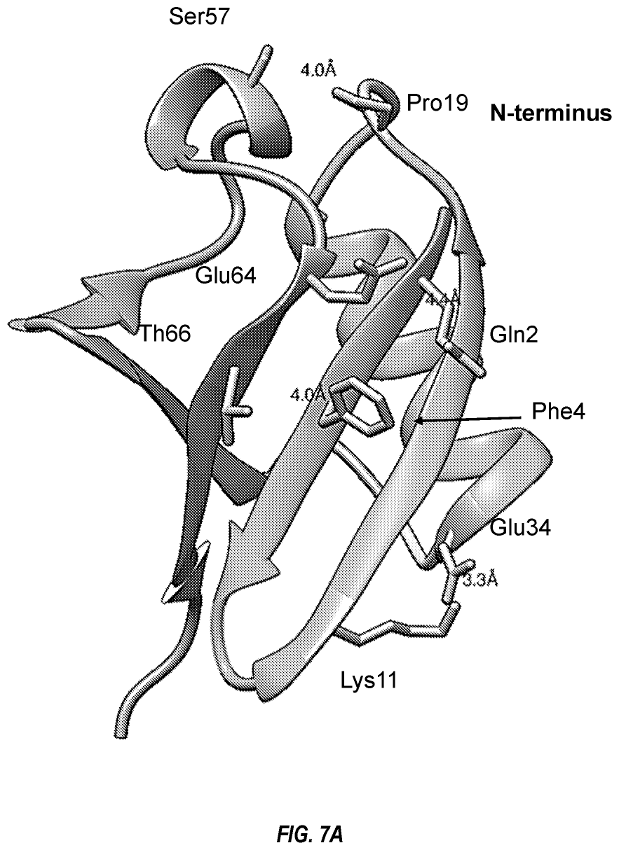

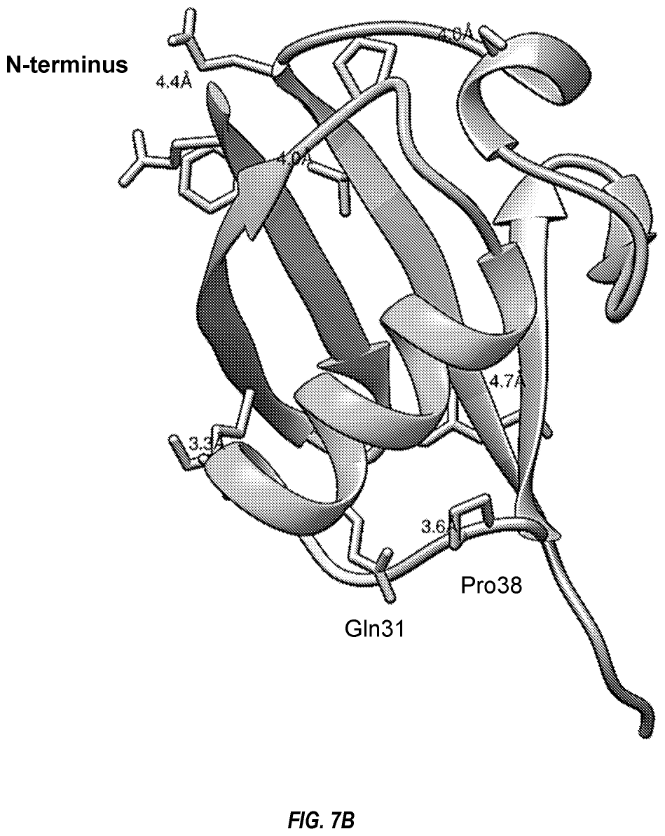

To further improve the blocking ability of a split ubiquitin mask, point mutations were made along interacting surfaces of the two halves of ubiquitin based on the crystal structure of the protein (FIG. 7A-7B). The point mutations were made to areas in ubiquitin that are close to one another to induce an electrostatic salt bridge interaction or a covalent disulfide linkage, which may improve the interaction between the two portions. Amino acid pairs that were substituted to induce these interactions included Q2 and E64, F4 and T66, K11 and E34, P19 and S57, and Q31 and P38.

Point mutations can also be included within a ubiquitin portion. For example, substitutions T66K and/or L56K may create one or more salt bridges with other residues in the N-terminal portion of the ubiquitin. Mutations corresponding to Q2K, V5L, V26F, V26L, and/or L43F may increase hydrophobicity of the core. In addition, pairs of amino acids corresponding to positions F4/T66, Ki 1/E34, P19/S57, and Q31/P38 of ubiquitin were mutated to cysteine on a sUb-masked-hB36H-12.3 antibody, to create disulfide bonds.

Specific point mutations are as described in Table 5 and Table 6.

TABLE 5
sUb Light Chain Variants

| sUb | | SEQ | Cleavage | Light Chain Mask (including | SEQ |
|---|---|---|---|---|---|
| Variant | Linker | ID NO: | Sequence | linker and cleavage sequence) | ID NO: |
| SUb-Q2K- | GGGS | 45 | IPV | MKIFVKTLTGKTITLEVEPSDTIENVKA | 19 |
| LC-IPV | | | | KIQDKEGIPGGGSIPVSLRSG | |
| SUb-Q2K- | GGSG | 50 | PLG | MKIFVKTLTGKTITLEVEPSDTIENVKA | 20 |
| LC-PLG | | | | KIQDKEGIPGGSGPLGLAGGS | |
| SUb-Q2K- | GGSG | 50 | ScR | MKIFVKTLTGKTITLEVEPSDTIENVKA | 21 |
| LC-ScR | | | | KIQDKEGIPGGSGLALGPGGS | |
| SUb-Q2R- | GGGS | 45 | IPV | MRIFVKTLTGKTITLEVEPSDTIENVKA | 22 |
| LC-IPV | | | | KIQDKEGIPGGGSIPVSLRSG | |
| sUb-K11C- | GGGS | 45 | IPV | MQIFVKTLTGCTITLEVEPSDTIENVKA | 23 |
| LC-IPV | | | | KIQDKEGIPGGGSIPVSLRSG | |
| sUb-P19C- | GGGS | 45 | IPV | MQIFVKTLTGKTITLEVECSDTIENVKA | 24 |
| LC-IPV | | | | KIQDKEGIPGGGSIPVSLRSG | |
| sUb-Q31K- | GGGS | 45 | IPV | MQIFVKTLTGKTITLEVEPSDTIENVKA | 25 |
| LC-IPV | | | | KIKDKEGIPGGGSIPVSLRSG | |
| sUb-Q31R- | GGGS | 45 | IPV | MQIFVKTLTGKTITLEVEPSDTIENVKA | 26 |
| LC-IPV | | | | KIRDKEGIPGGGSIPVSLRSG | |
| sUb-Q31C- | GGGS | 45 | IPV | MQIFVKTLTGKTITLEVEPSDTIENVKA | 27 |
| LC-IPV | | | | KICDKEGIPGGGSIPVSLRSG | |
| sUb-F4C- | GGGS | 45 | IPV | MQICVKTLTGKTITLEVEPSDTIENVKA | 28 |
| LC-IPV | | | | KIQDKEGIPGGGSIPVSLRSG | |
| SUb-F4E- | GGGS | 45 | IPV | MQIEVKTLTGKTITLEVEPSDTIENVKA | 29 |
| LC-IPV | | | | KIQDKEGIPGGGSIPVSLRSG | |
| sUb-F4D- | GGGS | 45 | IPV | MQIDVKTLTGKTITLEVEPSDTIENVKA | 30 |
| LC-IPV | | | | KIQDKEGIPGGGSIPVSLRSG | |
| SUb-Q2K- | GGSG | 50 | PLG | MKIFVKTLTGKTITLEVEPSDTIENFKA | 31 |
| V26F-LC- | | | | KIQDKEGIPGGSGPLGLAGGS | |
| PLG | | | | | |
| sUb-Q2K- | GGSG | 50 | PLG | MKIFLKTLTGKTITLEVEPSDTIENLKA | 32 |
| V5L-V26L- | | | | KIQDKEGIPGGSGPLGLAGGS | |
| LC-PLG | | | | | |

TABLE 6
sUb Heavy Chain Variants

| sUb | | SEQ | Cleavage | Heavy Chain Mask (including | SEQ |
|---|---|---|---|---|---|
| Variant | Linker | ID NO: | Sequence | linker and cleavage sequence) | ID NO: |
| sUb-E34D- | GSGGS | 44 | IPV | GDGIPPDQQRLIFAGKQLEDGRTLSDYNI | 33 |
| HC-IPV | | | | QKESTLHLVLRLGSGGSIPVSLRSG | |
| sUb-E34C- | GSGGS | 44 | IPV | GCGIPPDQQRLIFAGKQLEDGRTLSDYNI | 34 |
| HC-IPV | | | | QKESTLHLVLRLGSGGSIPVSLRSG | |
| SUb-S57C- | GSGGS | 44 | IPV | GIPPDQQRLIFAGKQLEDGRTLCDYNIQK | 35 |
| HC-IPV | | | | ESTLHLVLRLGSGGSIPVSLRSG | |
| sUb-P38D- | GSGGS | 44 | IPV | GIPDDQQRLIFAGKQLEDGRTLSDYNIQK | 36 |
| HC-IPV | | | | ESTLHLVLRLGSGGSIPVSLRSG | |
| sUb-P38C- | GSGGS | 44 | IPV | GIPCDQQRLIFAGKQLEDGRTLSDYNIQK | 37 |
| HC-IPV | | | | ESTLHLVLRLGSGGSIPVSLRSG | |
| sUb-P38E- | GSGGS | 44 | IPV | GIPEDQQRLIFAGKQLEDGRTLSDYNIQK | 38 |
| HC-IPV | | | | ESTLHLVLRLGSGGSIPVSLRSG | |
| sUb-T66C- | GSGGS | 44 | IPV | GIPPDQQRLIFAGKQLEDGRTLSDYNIQK | 39 |
| HC-IPV | | | | ESCLHLVLRLGSGGSIPVSLRSG | |
| SUb-T66K- | GSGGS | 44 | IPV | GIPPDQQRLIFAGKQLEDGRTLSDYNIQK | 40 |
| HC-IPV | | | | ESKLHLVLRLGSGGSIPVSLRSG | |
| SUb-T66R- | GSGGS | 44 | IPV | GIPPDQQRLIFAGKQLEDGRTLSDYNIQK | 41 |
| HC-IPV | | | | ESRLHLVLRLGSGGSIPVSLRSG | |
| SUb-T66K- | GSGGS | 44 | IPV | GIPPDQQRLIFAGKQLEDGRTKSDYNIQK | 42 |
| L56K-HC- | | | | ESKLHLVLRLGSGGSIPVSLRSG | |
| IPV | | | | | |
| SUb-L43F- | GGSG | 50 | PLG | GIPPDQQRFIFAGKQLEDGRTLSDYNIQK | 43 |
| HC-PLG | | | | ESTLHLVLRLGGSGPLGLAGGS | |

Binding was assessed on CD47-expressing SW780 cells via flow cytometry (FIG. 8). Masked antibody comprising sUb-P19C-LC-IPV and sUb-S57C-HC-IPV and masked antibody comprising sUb-Q31C-LC-IPV and sUb-P38C-HC-IPV each reduced affinity of the masked antibody by ˜100-fold compared to the unmasked hB6H12.3 antibody. Masked antibody comprising sUb-F4C-LC-IPV and sUb-T66C-HC-IPV and masked antibody comprising sUb-K11C-LC-IPV and sUb-E34C-HC-IPV each reduced the affinity of the masked antibody by >100 fold compared to unmasked-hB6H12.3.

Amino acid pairs at positions corresponding to position Q2K (to bridge with E64), as well as at pairs of positions corresponding to F4/T66, K11/E34, P19/S57, and Q31/P38 of ubiquitin were also assessed for the blocking ability of sUb. The amino acids at the indicated positions were mutated to either a cationic amino acid (lysine or arginine) or an anionic amino acid (glutamic acid or aspartic acid) and paired to create a salt bridge. sUb-masked chains containing these mutations were fused to each of hB6H12.3, hB6H12.3-low affinity, and Antibody A-low affinity antibodies. Binding was assessed using flow cytometry on antigen-expressing cells as described generally in Example 6.

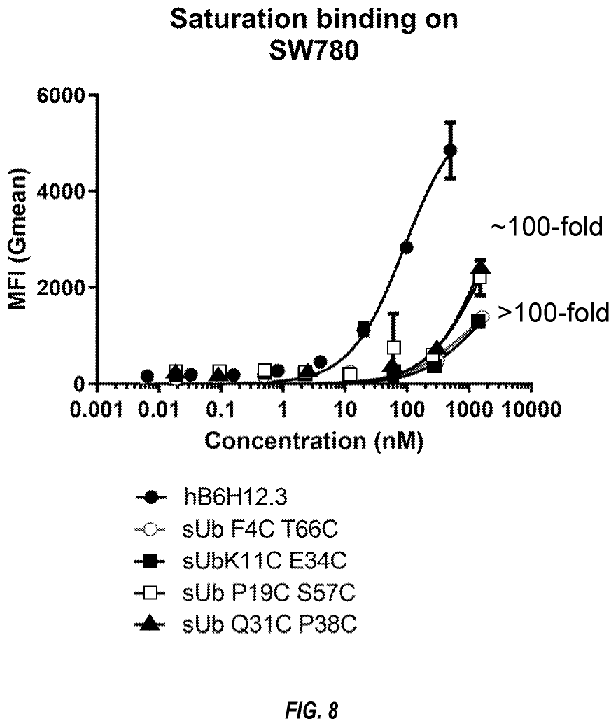

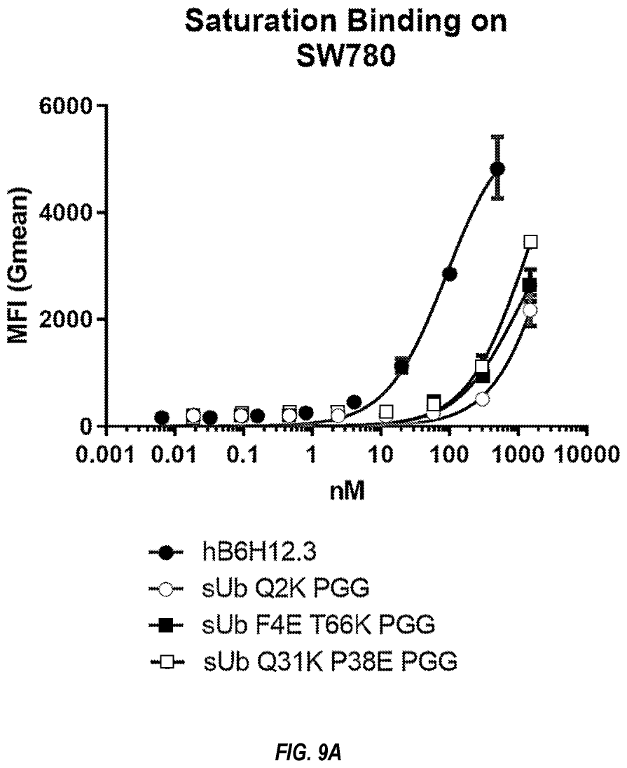

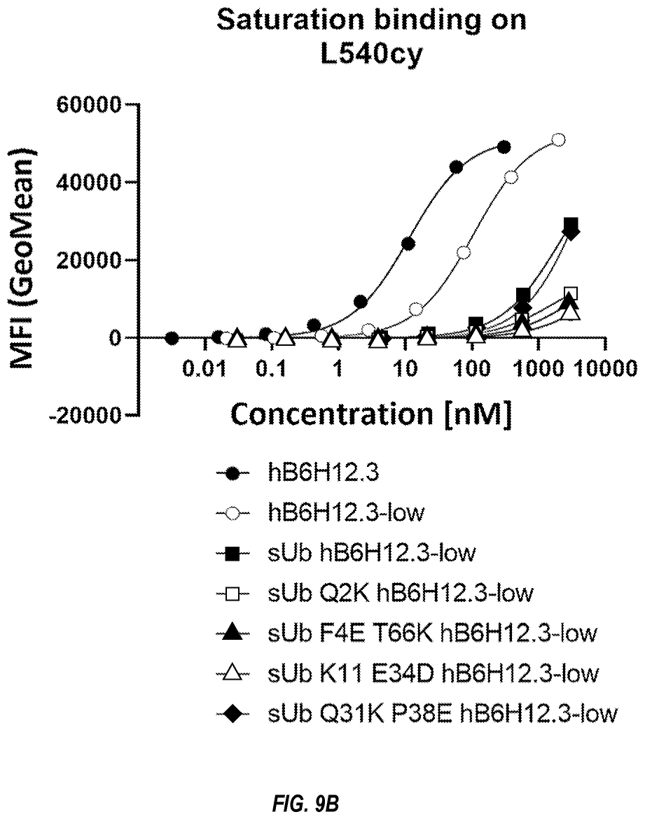

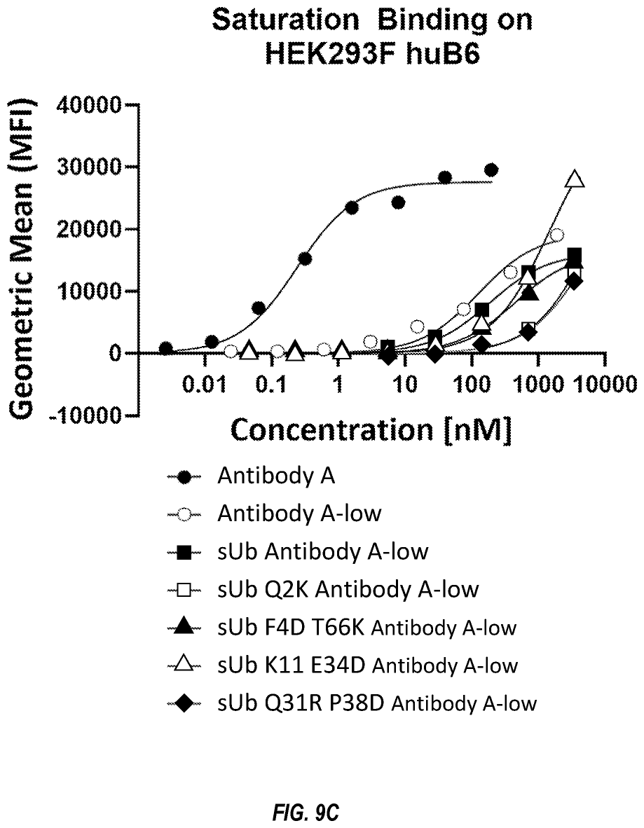

For sUb-masked-hB6H12.3, masked antibody having mutations sUb-Q2K-LC-IPV (and heavy chain mask sUb-HC-IPV) and masked antibody having sUb-F4E-LC-IPV/sUb-T66K-HC-IPV did not reach saturation for concentrations as high as 1.5 μM whereas masked antibody having sUb-F4E-LC-IPV/sUb-T66K-HC-IPV reduced the affinity of the sUb-masked antibody by approximately 10-fold compared to unmasked hB6H12.3 (FIG. 9A).

The hB6H12.3-low affinity antibody has a EC₅₀ of 105 nM and the sUb-masked antibody having sUb-Q2K-LC-IPV (and heavy chain mask sUb-HC-IPV), masked antibody having sUb-F4E-LC-IPV/sUb-T66K-HC-IPV, as well as masked antibody having sUb-LC-IPV/sUb-E34D-HC-IPV resulted in minimal binding for concentrations as high as 3 μM, resulting in a reduction in affinity of greater than 30-fold compared to unmasked hB6H12.3-low affinity antibody. Masked antibody having sUb-LC-IPV/sUb-HC-IPV and masked antibody having sUb-Q31K-LC-IPV/sUb-P38E-HC-IPV reduced binding by approximately 30-fold compared to unmasked hB6H12.3-low affinity antibody (FIG. 9B).

For the Antibody A-low affinity antibody, masked antibody having sUb-Q2K-LC-IPV (and heavy chain mask sUb-HC-IPV) and the sUb-masked antibody having sUb-Q31R-LC-IPV/sUb-P38D-HC-IPV masked the most effectively, whereas the sUb-masked antibody having sUb-F4-LC-IPV/sUb-T66-HC-IPV and sUb-LC-IPV/sUb-E34D-HC-IPV of ubiquitin were less effective (FIG. 9C).

Based on these results, it appeared the Q2K substitution resulted in the most consistent improvement in blocking of the sUb mask.

### Example 8: Preparation of Masked Antibody Drug Conjugates (ADCs)

Antibody interchain disulfides were partially reduced using TCEP (12 mol. equiv., pH 8, 37° C., 90 minutes) in the presence of 1 mM DTPA until 8 reduced thiols were present. Excess TCEP and DTPA were removed via buffer exchange with Amicon centrifugal filters into PBS containing 2 mM EDTA. Reduced antibodies were conjugated for 15 minutes at RT with a 1.5-fold molar excess per thiol of MDpr-glucuronide-MMAE-maleimide or MDpr-glucuronide-AT-maleimide from DMSO stock solutions. The conjugation reaction was purified using a Nap5 column and buffer exchanged into PBS as described above. Drug-loading was determined by LC-MS.

### Example 9: Viability Assay

Cell viability assays were performed using CellTiter-Glo (Promega). BxPC3, Detroit 562, HPAF-II, SW780, and HCT-116 cells (2000 cells/well) were seeded into a 96-well clear bottom culture plates. ADC dilutions were added to each well (1000 ng/mL to 0 ng/mL) and the samples were incubated for 96 hours at 37° C. Luminescence was measured using an an EnVision Multilabel Plate Reader (Perkin Elmer). The IC50 value was determined in quadruplicate and is defined as the concentration that results in half maximal growth inhibition over the course of the titration curve. The data was fit using GraphPad Prism6 and an IC50 was used to compare the change in cytotoxicity of parent ADC against masked ADC.

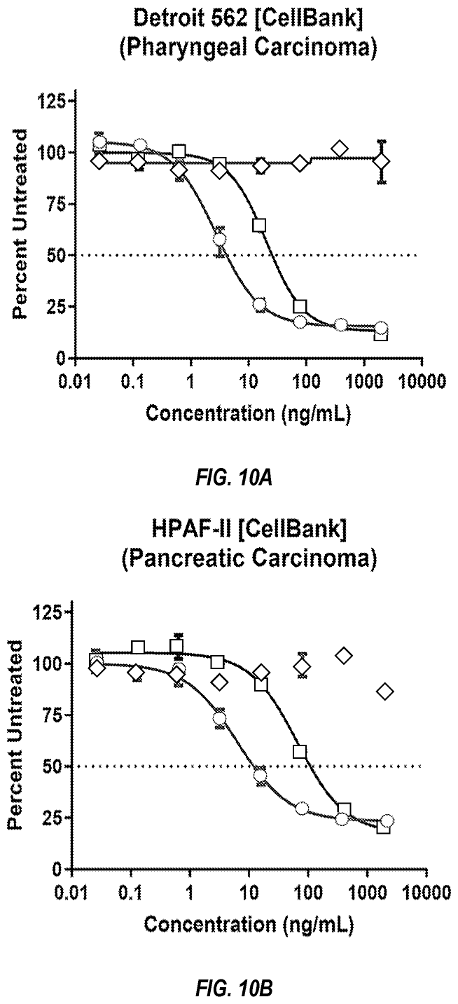

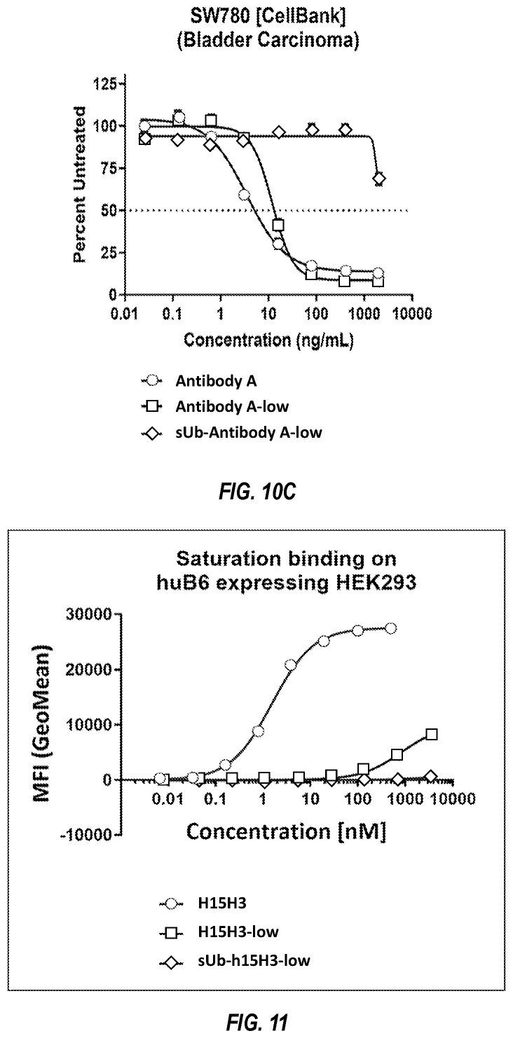

The effect of masking on in vitro cytotoxicity was evaluated by comparing sUb-masked with parent integrin αvβ6 binding antibody, Antibody A-low affinity (FIG. 10A-10C). Parent Antibody A-low and a sUb-masked Antibody A-low version having sUb-HC-IPV/sUb-LC-IPV heavy and light chain antibody drug conjugates (ADCs), both bearing an auristatin-based drug linker, gluc-MMAE with a drug-antibody ratio (DAR) of about eight were incubated for 96 hours with αvβ6-expressing cell lines: BxPC3, Detroit 562, HPAF-II, SW780, and αvβ6-negative HCT-116. The parent Antibody A-low ADC had IC50 values ranging from 22-108 ng/mL whereas the sUb-masked ADC had no activity for concentrations up to 2 μg/mL, as shown in Table 7.

TABLE 7

| | | BxPC-3 | Detroit562 | HPAF-II | |
|---|---|---|---|---|---|
| | | (37K) | (43K) | (22K) | SW780 |
| | | IC₅₀ | IC₅₀ | IC₅₀ | IC₅₀ |
| ADC/Drug | Drug | (ng/mL) | (ng/mL) | (ng/mL) | (ng/mL) |
| Antibody A | MDpr-Gluc- | 5 | 4 | 12 | 5 |
| | PEG12- | | | | |
| | MMAE | | | | |
| Antibody | MDpr-Gluc- | 22 | 26 | 108 | 13 |
| A-low | PEG12- | | | | |
| | MMAE | | | | |
| sUb | MDpr-Gluc- | >2K | >2K | >2K | >2K |
| Antibody | PEG12- | | | | |
| A-low | MMAE | | | | |

These results demonstrate that the sUb mask is able to prevent binding and cell-killing for concentrations up to 2 μg/mL.

The effect of masking on in vitro cytotoxicity was also evaluated using integrin αvβ6 binding antibody, h15H3-low affinity (FIG. 11). Parent h15H3-low and a sUb-masked h15H3-low version having sUb-LC-IPV/sUb-HC-IPV masked antibody drug conjugates (ADCs) bearing an auristatin-based drug linker, auristatin-based drug linker with a drug-antibody ratio (DAR) of about eight were incubated for 96 hours with αvβ6-expressing cell lines: BxPC3, Detroit 562, HPAF-II, SW780, with results as shown in Table 8.

TABLE 8

| ADC/Drug | BxPC-3 | Detroit562 | HPAFII | SW780 |
|---|---|---|---|---|
| h15H3 | 1 | 58 | 23 | 16 |
| h15H3-low | 35 | 1114 | 133 | 114 |
| sUb-IPV-h15H3-low | 2136 | 15824 | 4835 | 3488 |

Compared to the unmasked h15H3-low ADCs, the sUb-h15H3-low reduced the IC50 of the ADCs by a factor of 14-60 fold. This reduction in activity corresponds with the reduction in binding with the sUb mask.

### Example 10: In Vivo Efficacy Studies

All experiments were conducted in concordance with the Institutional Animal Care and Use Committee in a facility full accredited by the Association and Accreditation of Laboratory Animal Care.

For therapy experiments, 5×10⁶ cells were injected subcutaneously into 5-8 female nude mice (Envigo) for the BxPC3 and HPAF-II studies. Mice were randomly divided to study groups and dosed with test article via intraperitoneal injection once the tumors reached approximately 100 mm³. Animals were euthanized when tumor volumes reached 500-1000 mm³. Tumor volume was calculated with the formula (volume=12×length×width×width). Mice were terminated around day 31-56 after implant. In all xenograft studies, no weight loss or treatment-related toxicities were observed for mice treated with any of the test articles.

### Example 11: Quantification of Cleaved Antibody from Xenograft Studies

Frozen tissues obtained from mice dosed with antibodies were homogenized into powder on dry ice by physical disruption. The homogenized tissue was then resuspended into T-PER homogenization solution (Thermo Scientific) containing protease inhibitor cocktail using a ratio of tissue homogenate to protease inhibitor based on T-PER product instructions. Human IgG specific affinity resin, IgSelect (GE Healthcare Life Sciences), was used to capture antibodies from the tissue homogenates by incubation at 4° C. overnight. The antibodies were eluted from the resin using 50 mM glycine buffer, pH 3, and concentrated using an Amicon Ultra 30 kDa spin filter to a volume of ca. 30 μL. Purified antibodies were separated on a 12% Tris glycine gel (ThermoFisher) under reduced denaturing conditions, then transferred onto nitrocellulose membranes. The masked and cleaved antibody signals were detected using an H1RP-conjugated anti-human IgG (H+L) antibody (Jackson ImmunoResearch). The blots were scanned using an Amersham Imager 600 (GE Healthcare Life Sciences) and band intensities were quantified with ImageJ software (NIH). The percent of cleaved antibody signal was calculated as cleaved signal divided by total signal of cleaved and uncleaved antibody.

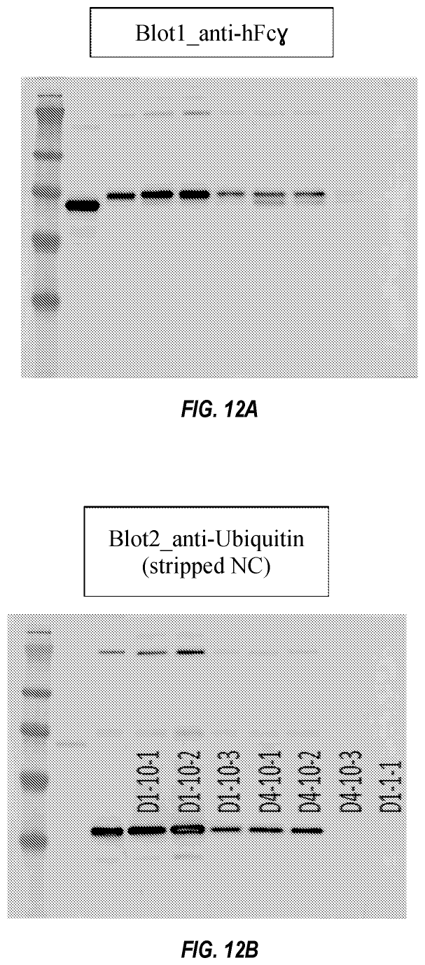

Because sUb is an important post-translational modification that is prone to cleavage and ubiquitination, the stability of sUb mask in vivo was assessed. sUb-Antibody A-low-gluc MMAE ADC having sUb-LC-IPV/sUb-HC-IPV masks was injected into Nude mice via intravenous administration at a dose of 3 mg/kg. Plasma was assessed via western blot at day 1 and day 4. By day 4, the majority of C-terminal half of the sUb mask was removed on the heavy chain (FIG. 12). It is unclear if this cleavage is due to deubiquitinases or other proteases. To assess the ubiquitination state of the masked antibody, the western blot was further probed using anti-ubiquitin. sUb-Antibody A-low does not serve as a substrate for further ubiquitination given the lack of higher molecular weight species on the heavy and light chain of the antibody as shown by the anti hFc and anti-ubiquitin blots.

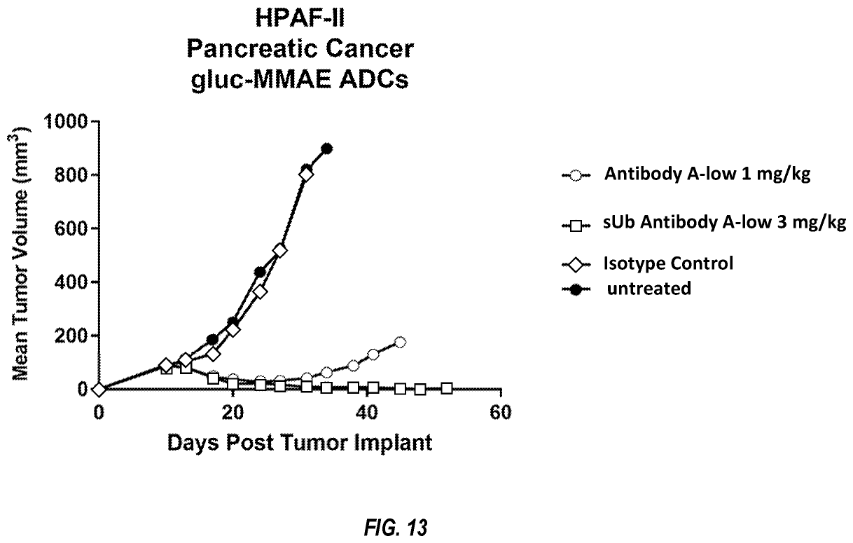

To evaluate the activation of masked Antibody A-low ADCs in tumors, the activity of unmasked Antibody A-low and sUb-Antibody A-low gluc-MMAE ADCs in αvβ6-expressing HPAF-II pancreatic cancer xenograft tumor model in Nude mice was tested (FIG. 13). The mice were dosed with 1 mg/kg of Antibody A-low and 3 mg/kg of sUb-Antibody A-low once tumors reached 100 mm³. Despite having a ˜20-fold difference in activity in vitro, in vivo, sUb-Antibody A-low has approximately 3-fold less activity than the unmasked ADC, which suggests that the mask is cleaved from the ADC leading to binding and uptake of the cell.

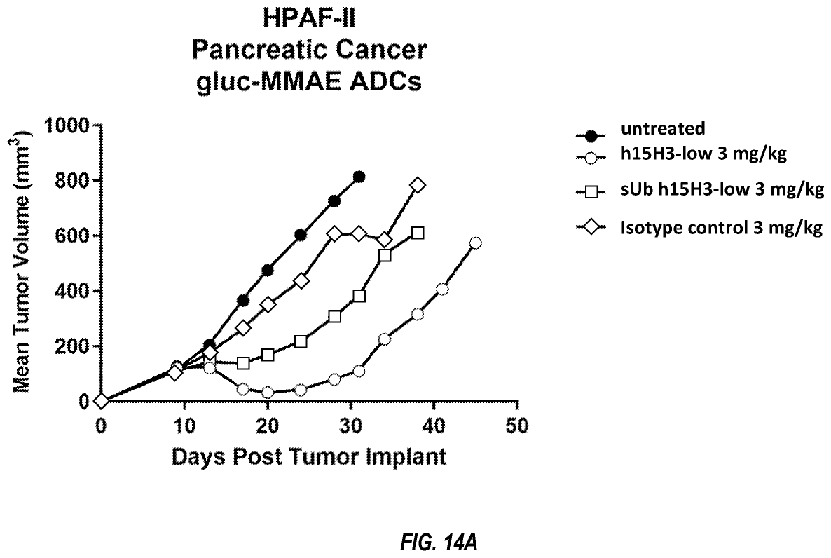

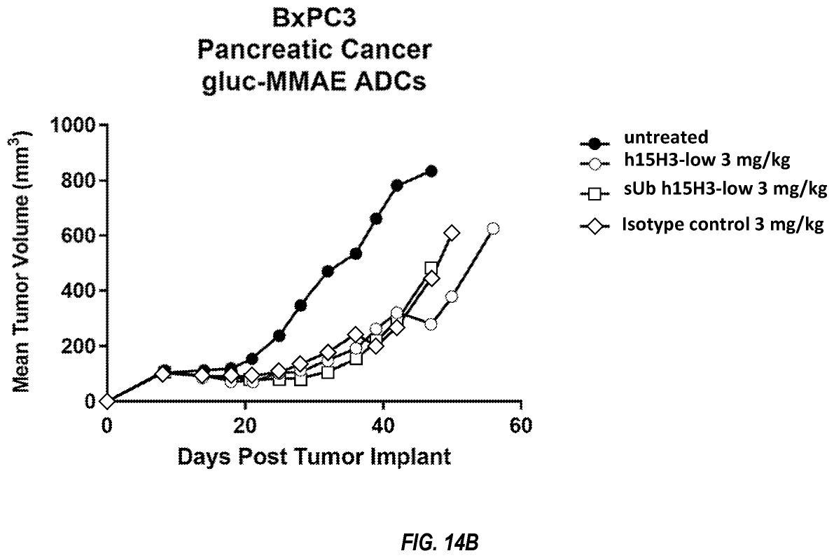

To appraise the activity of masked h15H3-low ADCs in tumors, the activity of unmasked h15H3-low and sUb-h15H3-low gluc-MMAE ADCs in αvβ6-expressing HPAF-II and BxPC3 pancreatic cancer xenograft tumor models in Nude mice was tested. The mice were dosed with 3 mg/kg ADC once tumors reached 100 mm³. In the HPAF-II model, sUb h15H3-low displays anti-tumor activity, but not to the same extent as the unmasked ADC. The decrease in activity may be attributed to the cleavage kinetics of the mask on h15H3-low (FIG. 14A). In the BxPC3 model, sUb h15H3-low displays similar anti-tumor activity to the unmasked ADC (FIG. 14B).

### Example 12: Rat-Toxicity Study

All experiments were conducted in concordance with the Institutional Animal Care and Use Committee in a facility full accredited by the Association and Accreditation of Laboratory Animal Care.

Sprague Dawley rats (Envigo, n=3) were dosed with test article intravenously. At 4 days post-dose animals were euthanized and necropsied. Histologic evaluation and processing were performed at Charles River Laboratories.

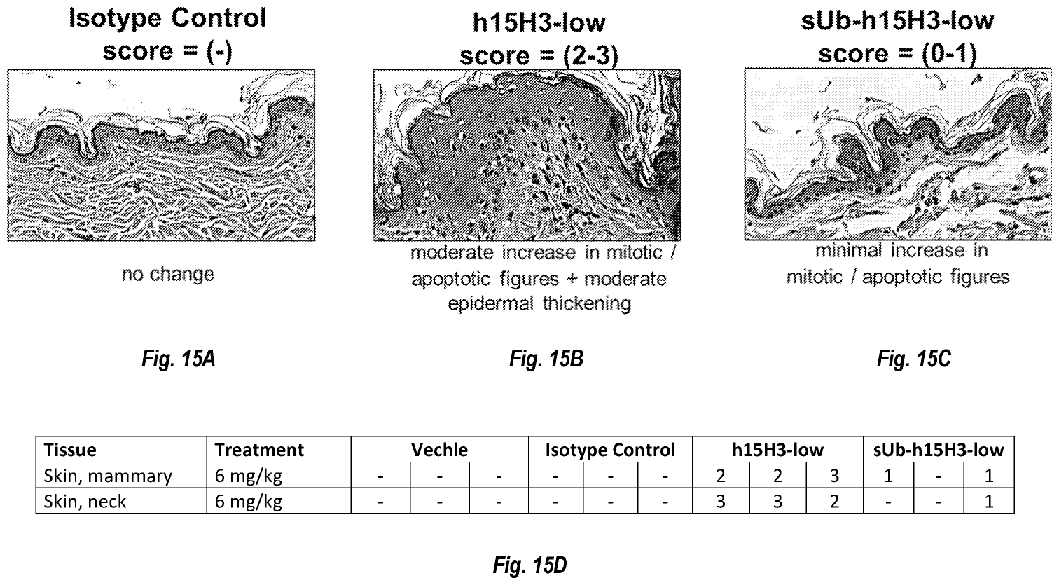

The role of the mask toward the onset of on-target toxicity using αvβ6 rodent-cross reactive h15H3-low gluc-AT ADCs was evaluated (FIG. 15A-15D). Rats were dosed with, respectively, h15H3-low ADC and sUb-h15H3-low ADC (having sUb-LC-IPV/sUb-HC-IPV masks) intravenously and sacrificed 4 days post-dose. At 6 mg/kg in Sprague-Dawley rats, unmasked h15H3-low ADCs have moderate skin toxicities that result in increased mitotic/apoptopic figures and epidermal thickening. These events can be found in both mammary and neck regions. Administration of h15H3-low gluc-AT at 6 mg/kg resulted in mild skin changes with the score of 0-1 where there is minimal increase in mitotic/apoptotic figures. These results suggest that the masking strategy is able to reduce the on-target toxicity found in the rodent cross-reactive h15H3-low gluc-MMAE ADC.

The sUb mask has been modified to improve blocking, improved aggregation, should have lower immunogenicity. For the h15H3 ADCs, the sUb mask has reduced toxicity by 6-fold and improved the therapeutic window by 2. The sUb-mask potentially improved immunogenicity and behavior of the antibody it masked, especially if coupled with a reduced affinity antibody.

TABLE of
Certain Sequences

| SEQ | | |
|---|---|---|
| ID NO: | Description | Sequence |
| 1 | Ubiquitin | MQIFVKTLTG KTITLEVEPS DTIENVKAKI QDKEGIPPDQ QRLIFAGKQL |
| | | EDGRTLSDYN IQKESTLHLV LRLRGG |
| 2 | Amino acids | MQIFVKTLTG KTITLEVEPS DTIENVKAKI QDKEGIP |
| | 1-37 of | |
| | ubiquitin | |
| 3 | Amino acids | GIPPDQQRLI FAGKQLEDGR TLSDYNIQKE STLHLVLRLR GG |
| | 35-76 of | |
| | ubiquitin | |
| 4 | sUb-HC-PGG | GIPPDQQRLIFAGKQLEDGRTLSDYNIQKESTLHLVLRLPGGGS |
| 5 | sUb-HC-GS | GIPPDQQRLIFAGKQLEDGRTLSDYNIQKESTLHLVLRLGSGGS |
| 6 | sUb-LC-IPV | MQIFVKTLTGKTITLEVEPSDTIENVKAKIQDKEGIPGGGSIPVSLRSG |
| 7 | sUb-LC-PLG | MQIFVKTLTGKTITLEVEPSDTIENVKAKIQDKEGIPGGSGPLGLAGGS |
| 8 | sUb-LC-Scr | MQIFVKTLTGKTITLEVEPSDTIENVKAKIQDKEGIPGGSGLALGPGGS |
| 9 | sUbext-LC-PGG | MQIFVKTLTGKTITLEVEPSDTIENVKAKIQDKEGIPPDQQRLGGGS |
| 10 | sUbext-LC-IPV | MQIFVKTLTGKTITLEVEPSDTIENVKAKIQDKEGIPPDQQRLGGGSIPVSLRSG |
| 11 | sUb-LC-flip | GIPPDQQRLIFAGKQLEDGRTLSDYNIQKESTLHLVLRLPGGGGIPVSLRSG |
| 12 | sUb-HC-IPV | GIPPDQQRLIFAGKQLEDGRTLSDYNIQKESTLHLVLRLGSGGSIPVSLRSG |
| 13 | sUb-HC-PLG | GIPPDQQRLIFAGKQLEDGRTLSDYNIQKESTLHLVLRLGGSGPLGLAGGS |
| 14 | sUb-HC-Scr | GIPPDQQRLIFAGKQLEDGRTLSDYNIQKESTLHLVLRLGGSGLALGPGGS |
| 15 | sUbext-HC-PGG | GIPPDQQRLIFAGKQLEDGRTLSDYNIQKESTLHLVLRLPGGGS |
| 16 | sUbext-HC-IPV | GIPPDQQRLIFAGKQLEDGRTLSDYNIQKESTLHLVLRLPGGGSIPVSLRSG |
| 17 | sUb-HC-short | GIPPDQQRLIFAGKQLEDGRTLSDYNIQKESTLHLVLGGSGPLGLAGGS |
| 18 | sUb-HC-flip | MQIFVKTLTGKTITLEVEPSDTIENVKAKIQDKEGIPGGGGIPVSLRSG |
| 19 | sUb-Q2K- | MKIFVKTLTGKTITLEVEPSDTIENVKAKIQDKEGIPGGGSIPVSLRSG |
| | LC-IPV | |
| 20 | sUb-Q2K- | MKIFVKTLTGKTITLEVEPSDTIENVKAKIQDKEGIPGGSGPLGLAGGS |
| | LC-PLG | |
| 21 | sUb-Q2K- | MKIFVKTLTGKTITLEVEPSDTIENVKAKIQDKEGIPGGSGLALGPGGS |
| | LC-ScR | |
| 22 | sUb-Q2R- | MRIFVKTLTGKTITLEVEPSDTIENVKAKIQDKEGIPGGGSIPVSLRSG |
| | LC-IPV | |
| 23 | sUb-K11C- | MQIFVKTLTGCTITLEVEPSDTIENVKAKIQDKEGIPGGGSIPVSLRSG |
| | LC-IPV | |
| 24 | sUb-P19C- | MQIFVKTLTGKTITLEVECSDTIENVKAKIQDKEGIPGGGSIPVSLRSG |
| | LC-IPV | |
| 25 | sUb-Q31K- | MQIFVKTLTGKTITLEVEPSDTIENVKAKIKDKEGIPGGGSIPVSLRSG |
| | LC-IPV | |
| 26 | sUb-Q31R- | MQIFVKTLTGKTITLEVEPSDTIENVKAKIRDKEGIPGGGSIPVSLRSG |
| | LC-IPV | |
| 27 | sUb-Q31C- | MQIFVKTLTGKTITLEVEPSDTIENVKAKICDKEGIPGGGSIPVSLRSG |
| | LC-IPV | |
| 28 | sUb-F4C-LC- | MQICVKTLTGKTITLEVEPSDTIENVKAKIQDKEGIPGGGSIPVSLRSG |
| | IPV | |
| 29 | sUb-F4E-LC- | MQIEVKTLTGKTITLEVEPSDTIENVKAKIQDKEGIPGGGSIPVSLRSG |
| | IPV | |
| 30 | sUb-F4D- | MQIDVKTLTGKTITLEVEPSDTIENVKAKIQDKEGIPGGGSIPVSLRSG |
| | LC-IPV | |
| 31 | sUb-Q2K- | MKIFVKTLTGKTITLEVEPSDTIENFKAKIQDKEGIPGGSGPLGLAGGS |
| | V26F-LC-PLG | |
| 32 | sUb-Q2K- | MKIFLKTLTGKTITLEVEPSDTIENLKAKIQDKEGIPGGSGPLGLAGGS |
| | V5L-V26L- | |
| | LC-PLG | |
| 33 | sUb-E34D- | GDGIPPDQQRLIFAGKQLEDGRTLSDYNIQKESTLHLVLRLGSGGSIPVSLRSG |
| | HC-IPV | |
| 34 | sUb-E34C- | GCGIPPDQQRLIFAGKQLEDGRTLSDYNIQKESTLHLVLRLGSGGSIPVSLRSG |
| | HC-IPV | |
| 35 | sUb-S57C- | GIPPDQQRLIFAGKQLEDGRTLCDYNIQKESTLHLVLRLGSGGSIPVSLRSG |
| | HC-IPV | |
| 36 | sUb-P38D- | GIPDDQQRLIFAGKQLEDGRTLSDYNIQKESTLHLVLRLGSGGSIPVSLRSG |
| | HC-IPV | |
| 37 | sUb-P38C- | GIPCDQQRLIFAGKQLEDGRTLSDYNIQKESTLHLVLRLGSGGSIPVSLRSG |
| | HC-IPV | |
| 38 | sUb-P38E- | GIPEDQQRLIFAGKQLEDGRTLSDYNIQKESTLHLVLRLGSGGSIPVSLRSG |
| | HC-IPV | |
| 39 | SUb-T66C- | GIPPDQQRLIFAGKQLEDGRTLSDYNIQKESCLHLVLRLGSGGSIPVSLRSG |
| | HC-IPV | |
| 40 | sUb-T66K- | GIPPDQQRLIFAGKQLEDGRTLSDYNIQKESKLHLVLRLGSGGSIPVSLRSG |
| | HC-IPV | |
| 41 | sUb-T66R- | GIPPDQQRLIFAGKQLEDGRTLSDYNIQKESRLHLVLRLGSGGSIPVSLRSG |
| | HC-IPV | |
| 42 | sUb-T66K- | GIPPDQQRLIFAGKQLEDGRTKSDYNIQKESKLHLVLRLGSGGSIPVSLRSG |
| | L56K-HC-IPV | |
| 43 | sUb-L43F- | GIPPDQQRFIFAGKQLEDGRTLSDYNIQKESTLHLVLRLGGSGPLGLAGGS |
| | HC-PLG | |
| 44 | Linker 1 | GSGGS |
| 45 | Linker 2 | GGGS |
| 46 | Linker 3 | SGGGGGGGGGGS |
| 47 | Linker 4 | GGAA |
| 48 | Linker 5 | GGGGS |
| 49 | Linker 6 | LAAAA |
| 50 | Linker 7 | GGSG |
| 51 | Linker 8 | GGSGG |
| 52 | Linker 9 | GSGSG |
| 53 | Linker 10 | GSGGG |
| 54 | Linker 11 | GGGSG |
| 55 | Linker 12 | GSSSG |
| 56 | MMP | PLGXXX |
| | cleavage site | |
| 57 | IPV cleavage | IPVSLRSG |
| | site | |
| 58 | Linker-IPV | GSIPVSLRSG |
| | cleavage site | |
| 59 | M2 cleavage | GPLGVR |
| | site | |
| 60 | MMP | LSGRSDNY |
| | cleavage site | |
| | 1 | |
| 61 | MMP | PLGVR |
| | cleavage site | |
| | 2 | |
| 62 | Protease site | PLGLAG |
| | 1 | |
| 63 | Protease- | LALGPG |
| | resistant site | |
| 64 | Protease site | YGRAA |
| | 2 | |
| 65 | Stub | LRSG |
| | sequence | |
| 66 | sUb-PGG | GIPPDQQRLIFAGKQLEDGRTLSDYNIQKESTLHLVLRLPGG |
| | mask | |
| 67 | sUb mask 1 | GIPPDQQRLIFAGKQLEDGRTLSDYNIQKESTLHLVLRL |
| 68 | sUb | MQIFVKTLTGKTITLEVEPSDTIENVKAKIQDKEGIP |
| | mask 2 | |
| 69 | sUb-ext | MQIFVKTLTGKTITLEVEPSDTIENVKAKIQDKEGIPPDQQRL |
| | mask | |
| 70 | sUb-short | GIPPDQQRLIFAGKQLEDGRTLSDYNIQKESTLHLVL |
| | mask | |
| 71 | sUb-flip | MQIFVKTLTGKTITLEVEPSDTIENVKAKIQDKEGIPGG |
| | mask | |
| 72 | sUb-Q2K | MKIFVKTLTGKTITLEVEPSDTIENVKAKIQDKEGIP |
| | mask | |
| 73 | sUb-Q2R | MRIFVKTLTGKTITLEVEPSDTIENVKAKIQDKEGIP |
| | mask | |
| 74 | sUb-K11C- | MQIFVKTLTGCTITLEVEPSDTIENVKAKIQDKEGIP |
| | mask | |
| 75 | sUb-P19C | MQIFVKTLTGKTITLEVECSDTIENVKAKIQDKEGIP |
| | mask | |
| 76 | sUb-Q31K | MQIFVKTLTGKTITLEVEPSDTIENVKAKIKDKEGIP |
| | mask | |
| 77 | sUb-Q31R | MQIFVKTLTGKTITLEVEPSDTIENVKAKIRDKEGIP |
| | mask | |
| 78 | sUb-Q31C | MQIFVKTLTGKTITLEVEPSDTIENVKAKICDKEGIP |
| | mask | |
| 79 | sUb-F4C | MQICVKTLTGKTITLEVEPSDTIENVKAKIQDKEGIP |
| | mask | |
| 80 | sUb-F4E | MQIEVKTLTGKTITLEVEPSDTIENVKAKIQDKEGIP |
| | mask | |
| 81 | sUb-F4D | MQIDVKTLTGKTITLEVEPSDTIENVKAKIQDKEGIP |
| | mask | |
| 82 | sUb-Q2K- | MKIFVKTLTGKTITLEVEPSDTIENFKAKIQDKEGIP |
| | V26F mask | |
| 83 | sUb-Q2K- | MKIFLKTLTGKTITLEVEPSDTIENLKAKIQDKEGIP |
| | V5L-V26L | |
| | mask | |
| 84 | sUb-E34D | GDGIPPDQQRLIFAGKQLEDGRTLSDYNIQKESTLHLVLRL |
| | mask | |
| 85 | sUb-E34C | GCGIPPDQQRLIFAGKQLEDGRTLSDYNIQKESTLHLVLRL |
| | mask | |
| 86 | sUb-S57C | GIPPDQQRLIFAGKQLEDGRTLCDYNIQKESTLHLVLRL |
| | mask | |
| 87 | sUb-P38D | GIPDDQQRLIFAGKQLEDGRTLSDYNIQKESTLHLVLRL |
| | mask | |
| 88 | sUb-P38C | GIPCDQQRLIFAGKQLEDGRTLSDYNIQKESTLHLVLRL |
| | mask | |
| 89 | sUb-P38E | GIPEDQQRLIFAGKQLEDGRTLSDYNIQKESTLHLVLRL |
| | mask | |
| 90 | sUb-T66C | GIPPDQQRLIFAGKQLEDGRTLSDYNIQKESCLHLVLRL |
| | mask | |
| 91 | sUb-T66K | GIPPDQQRLIFAGKQLEDGRTLSDYNIQKESKLHLVLRL |
| | mask | |
| 92 | sUb-T66R | GIPPDQQRLIFAGKQLEDGRTLSDYNIQKESRLHLVLRL |
| | mask | |
| 93 | sUb-T66K- | GIPPDQQRLIFAGKQLEDGRTKSDYNIQKESKLHLVLRL |
| | L56K mask | |
| 94 | sUb-L43F | GIPPDQQRFIFAGKQLEDGRTLSDYNIQKESTLHLVLRL |
| | mask | |

What is claimed:

1. A masked antibody or antigen-binding fragment thereof comprising a first masking domain and a second masking domain, wherein the first masking domain comprises a portion of a ubiquitin or ubiquitin variant fused to a heavy chain variable region (VH) of an antibody, and the second masking domain comprises a portion of a ubiquitin or ubiquitin variant fused to a light chain variable region (VL) of the antibody.

2. The masked antibody or antigen-binding fragment of claim 1, comprising an N-terminal portion of a ubiquitin or ubiquitin variant and a C-terminal portion of a ubiquitin or ubiquitin variant.

3. The masked antibody or antigen-binding fragment of claim 2, wherein the N-terminal portion of the ubiquitin or ubiquitin variant and the C-terminal portion of the ubiquitin or ubiquitin variant have an overlap of 0-10 amino acids, such as 0 amino acids, 1 amino acid, 2 amino acids, 3 amino acids, 4 amino acids, 5 amino acids, 6 amino acids, 7 amino acids, 8 amino acids, 9 amino acids, or 10 amino acids compared to a full-length ubiquitin.

4. The masked antibody or antigen-binding fragment of any of the preceding claims, wherein the N-terminus of the VH is fused to a C-terminal portion of a ubiquitin or ubiquitin variant, and the N-terminus of the VL is fused to a N-terminal portion of a ubiquitin or ubiquitin variant.

5. The masked antibody or antigen-binding fragment of any of claims 1 to 3, wherein the N-terminus of the VH is fused to a N-terminal portion of a ubiquitin or ubiquitin variant, and the N-terminus of the VL is fused to a C-terminal portion of a ubiquitin or ubiquitin variant.

6. The masked antibody or antigen-binding fragment of any one of the preceding claims, wherein the N-terminal portion of the ubiquitin or ubiquitin variant and the C-terminal portion of the ubiquitin or ubiquitin variant block the binding of the antigen-binding domain to its antigen.

7. The masked antibody or antigen-binding fragment of any one of the preceding claims, wherein the N-terminal portion of the ubiquitin variant comprises one or more amino acid substitutions and/or a C-terminal extension compared to the corresponding portion of a wild-type ubiquitin.

8. The masked antibody or antigen-binding fragment of any one of the preceding claims, wherein the N-terminal portion of the ubiquitin or ubiquitin variant comprises at least 30 amino acid residues, such as at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 amino acid residues.

9. The masked antibody or antigen-binding fragment of any one of the preceding claims, wherein the N-terminal portion of the ubiquitin or ubiquitin variant comprises amino acids 1-37 of SEQ ID NO: 1.

10. The masked antibody or antigen-binding fragment of any one of the preceding claims, wherein the C-terminal portion of the ubiquitin or ubiquitin variant comprises at least 30 amino acid residues, such as at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 amino acid residues.

11. The masked antibody or antigen-binding fragment of any one of the preceding claims, wherein the C-terminal portion of the ubiquitin or ubiquitin variant comprises amino acids 35-76 of SEQ ID NO:1, optionally wherein a C-terminal fragment, such as 1 amino acid, 2 amino acids, 3 amino acids, 4 amino acids, 5 amino acids, or 6 amino acids, is removed.

12. The masked antibody or antigen-binding fragment of any one of the preceding claims, wherein the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 1, and wherein the variant comprises a modification at one or more positions corresponding to positions 2, 4, 5, 11, 19, 26, 31, 34, 38, 43, 56, 57, 64, 66, 74, 75, 76 of SEQ ID NO: 1, preferably wherein the modification is a substitution.

13. The masked antibody or antigen-binding fragment of any one of the preceding claims, wherein the first masking domain and the second masking domain comprise one or more interacting surfaces.

14. The masked antibody or antigen-binding fragment of claim 13, wherein the ubiquitin variant comprises one or more substitutions at one or more interacting surfaces of the first masking domain and the second masking domain.

15. The masked antibody or antigen-binding fragment of any one of the preceding claims, wherein the first masking domain and the second masking domain interact to form a globular structure.

16. The masked antibody or antigen-binding fragment of any one of the preceding claims, wherein the ubiquitin variant comprises one or more substitutions at a position corresponding to position Q2; positions Q2 and E64; positions F4 and T66; positions K11 and E34; positions P19 and S57; positions Q31 and P38; position L56; and/or position T66 of SEQ ID NO: 1.

17. The masked antibody or antigen-binding fragment of any one of the preceding claims, wherein the ubiquitin variant comprises one or more of, such as two or more of, three or more of, four or more of, or all five of the following pairs of substitutions: Q2C and E64C; F4C and T66C; K11C and E34C; P19C and S57C; and/or Q31C and P38C of SEQ ID NO: 1.

18. The masked antibody or antigen-binding fragment of any one of claims 1 to 16, wherein the ubiquitin variant comprises a substitution Q2D/E/K/R; F4D/E/K/R; K11D/E/R; P19D/E/K/R; Q31D/E/K/R; E34D/K/R; P38D/E/K/R; S57D/E/K/R; and/or T66D/E/K/R of SEQ ID NO: 1.

19. The masked antibody or antigen-binding fragment of any one of claims 1 to 16 or 18, wherein the ubiquitin variant comprises a substitution Q2K/R; a substitution E34D; a substitution L43F; a substitution L56K; a substitution T66K; and/or comprises a pair of substitutions F4D/E and T66K/R; K11R and E34D; and/or Q31R and P38D of SEQ ID NO: 1.

20. The masked antibody or antigen-binding fragment of any one of claims 1 to 16 or 18-19, wherein the ubiquitin variant comprises a substitution Q2D/E/K/R, such as Q2K or Q2R of SEQ ID NO: 1.

21. The masked antibody or antigen-binding fragment of any one of the preceding claims, wherein the N-terminal portion of the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 2, and wherein the variant comprises a modification at one or more positions corresponding to positions 2, 4, 5, 11, 19, 26, 31, of SEQ ID NO: 2, preferably wherein the modification is a substitution.

22. The masked antibody or antigen-binding fragment of any one of the preceding claims, wherein the ubiquitin variant comprises a substitution V5L of SEQ ID NO: 1 or SEQ ID NO: 2.

23. The masked antibody or antigen-binding fragment of any one of the preceding claims, wherein the ubiquitin variant comprises a substitution V26F/L of SEQ ID NO: 1 or SEQ ID NO: 2.

24. The masked antibody or antigen-binding fragment of any one of the preceding claims, wherein the C-terminal portion of the ubiquitin variant has at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% sequence identity to the amino acid sequence of SEQ ID NO: 3, and wherein the variant comprises a modification at one or more positions corresponding to positions 1, 4, 9, 40, 41, 42 SEQ ID NO: 3, preferably wherein the modification is a substitution.

25. The masked antibody or antigen-binding fragment of any one of the preceding claims, wherein the ubiquitin variant comprises a substitution L9F of SEQ ID NO: 3.

26. The masked antibody or antigen-binding fragment of any one of the preceding claims, wherein the ubiquitin variant comprises a substitution R40P of SEQ ID NO: 3.

27. The masked antibody or antigen-binding fragment of any one of the preceding claims, wherein the C-terminus of SEQ ID NO: 1, SEQ ID NO: 2 and/or SEQ ID NO: 3 comprises a linker, e.g., of the form S(G)nS, wherein n is from 5-20; or of the form (G)n, wherein n is an integer of at least one, such as Gly-Gly; or of the form (GS)n, wherein n is an integer of at least one, such as Gly-Ser; such as a linker of SEQ ID NOs: 44-55, such as SEQ ID NO: 44 or SEQ ID NO: 50.

28. The masked antibody or antigen-binding fragment of any one of the preceding claims, wherein the N-terminal portion of the ubiquitin or ubiquitin variant has at least 90% sequence identity, such as at least 92%, at least 93%, at least 94%, at least 95%, at least 96%, at least 97%, at least 98%, at least 99% or 100% sequence identity to any one of SEQ ID NOs: 2, 6-10, 18-32, 66-94.

29. The masked antibody or antigen-binding fragment of any one of the preceding claims, wherein the N-terminal portion of the ubiquitin or ubiquitin variant comprises or consists of any one of SEQ ID NOs: 2, 6-10, 18-32, 66-94.

30. The masked antibody or antigen-binding fragment of any one of the preceding claims, wherein the C-terminal portion of the ubiquitin or ubiquitin variant has at least 90% sequence identity, such as at least 92%, at least 93%, at least 94%, at least 95%, at least 96%, at least 97%, at least 98%, at least 99% or 100% sequence identity to any one of SEQ ID NOs: 3-5, 11-17, 33-43, 66-94.

31. The masked antibody or antigen-binding fragment of any one of the preceding claims, wherein the C-terminal portion of the ubiquitin or ubiquitin variant comprises or consists of any one of SEQ ID NOs: 3-5, 11-17, 33-43, 66-94.

32. The masked antibody or antigen-binding fragment of any one of the preceding claims, wherein the first masking domain and the second masking domain comprise the sequences of SEQ ID NOs: 2 and 4, respectively; or SEQ ID NOs: 2 and 5, respectively; or SEQ ID NOs: 6 and 12, respectively; or SEQ ID NOs: 7 and 13, respectively; or SEQ ID NOs: 8 and 14, respectively; or SEQ ID NOs: 9 and 15, respectively, or SEQ ID NOs: 10 and 16, respectively, or SEQ ID NOs: 7 and 17, respectively; or SEQ ID NOs: 11 and 18, respectively; or SEQ ID NOs: 19 and 12, respectively; or SEQ ID NOs: 20 and 13, respectively; or SEQ ID NOs: 21 and 14, respectively; or SEQ ID NOs: 22 and 12, respectively; SEQ ID NOs: 23 and 34, respectively; or SEQ ID NOs: 24 and 35, respectively; or SEQ ID NOs: 25 and 36, respectively; or SEQ ID NOs: 26 and 36, respectively; or SEQ ID NOs: 25 and 38, respectively; or SEQ ID NOs: 26 and 38, respectively; or SEQ ID NOs: 27 and 37, respectively; or SEQ ID NOs: 28 and 39, respectively; or SEQ ID NOs: 29 and 40, respectively; or SEQ ID NOs: 29 and 41, respectively; or SEQ ID NOs: 30 and 40, respectively; or SEQ ID NOs: 30 and 41, respectively; or SEQ ID NOs: 31 and 13, respectively; or SEQ ID NOs: 32 and 13, respectively; or SEQ ID NOs: 6 and 33, respectively; or SEQ ID NOs: 6 and 42, respectively; or SEQ ID NOs: 7 and 43, respectively.

33. The masked antibody or antigen-binding fragment of any one of the preceding claims, wherein each of the first masking domain and the second masking domain comprises a protease-cleavable linker and is linked to the heavy chain and the light chain via the protease-cleavable linker.

34. The masked antibody or antigen-binding fragment of claim 33, wherein the protease-cleavable linker of the first masking domain and the protease-cleavable linker of the second masking domain are the same.

35. The masked antibody or antigen-binding fragment of claim 33 or claim 34, wherein the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site.

36. The masked antibody or antigen-binding fragment of any one of claims 33-35, wherein the protease-cleavable linker comprises a matrix metalloprotease (MMP) cleavable sequence.

37. The masked antibody or antigen-binding fragment of claim 36, wherein the MMP cleavage site is selected from an MMP2 cleavage site, an MMP7 cleavage site, an MMP9 cleavage site and an MMP13 cleavage site.

38. The masked antibody or antigen-binding fragment of claim 36 or claim 37, wherein the MMP cleavage site comprises the sequence IPVSLRSG (SEQ ID NO: 57) or GPLGVR (SEQ ID NO: 59).

39. The masked antibody or antigen-binding fragment of any one of claims 36-38, wherein following cleavage by an MMP, the heavy chain and/or light chain of the antibody or antigen-binding fragment comprises a stub amino acid remnant of the MMP cleavage site.

40. The masked antibody or antigen-binding fragment of claim 39, wherein the stub amino acid remnant comprises the sequence LRSG (SEQ ID NO: 65), SG, or VR at the N terminus of the antibody.

41. The masked antibody or antigen-binding fragment of any of claims 1 to 32, wherein each of the first masking domain and the second masking domain comprises a protease-resistant linker and is linked to the heavy chain and/or the light chain via the protease-resistant linker.

42. The masked antibody or antigen-binding fragment of claim 41, wherein the protease-resistant linker comprises LALGPG (SEQ ID NO: 63).

43. The masked antibody or antigen-binding fragment of any one of the preceding claims, wherein the antibody or antigen-binding fragment binds a therapeutic antigen.

44. The masked antibody or antigen-binding fragment of claim 43, wherein the antibody is useful for treating cancer, an autoimmune disorder, or an infection.

45. The masked antibody or antigen-binding fragment of claim 43, wherein the antibody binds a tumor-associated antigen.

46. The masked antibody or antigen-binding fragment of claim 45, wherein the tumor-associated antigen is selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Axl, Clec12A, CD352, FAP, CXCR3, and CD5.

47. The masked antibody or antigen-binding fragment of any one of claims 1-46, wherein the masked antibody has reduced binding affinity for the antibody's target antigen compared to the binding affinity of the antibody for its target antigen without the first masking domain and second masking domain.

48. The masked antibody or antigen-binding fragment of claim 47, wherein the binding affinity is reduced at least about 30-fold compared to the antibody or antigen-binding fragment without the first masking domain and the second masking domain.

49. The masked antibody or antigen-binding fragment of claim 47 or claim 48, wherein the binding affinity is reduced at least about 50-fold compared to the antibody or antigen-binding fragment without the first masking domain and the second masking domain.

50. The masked antibody or antigen-binding fragment of any one of claims 47-49, wherein the binding affinity is reduced at least about 80-fold compared to the antibody or antigen-binding fragment without the first masking domain and the second masking domain.

51. The masked antibody or antigen-binding fragment of any one of claims 47-50, wherein the binding affinity is reduced at least about 100-fold compared to the antibody or antigen-binding fragment without the first masking domain and the second masking domain.

52. An immunoconjugate comprising the masked antibody or antigen-binding fragment of any one of claims 1-51 and a cytotoxic agent.

53. The immunoconjugate of claim 52, wherein the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer.

54. The immunoconjugate of claim 52 or claim 53, wherein the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid.

55. The immunoconjugate of any one of claims 52-54, wherein the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine.

56. The immunoconjugate of claim 55, wherein the cytotoxic agent is an auristatin.

57. The immunoconjugate of claim 55, wherein the cytotoxic agent is MMAE or MMAF.

58. A nucleic acid sequence encoding the masked antibody or antigen-binding fragment of any one of claims 1-51.

59. An expression vector comprising the nucleic acid of claim 58.

60. A host cell comprising the nucleic acid of claim 58 or the expression vector of claim 59.

61. A host cell that expresses the masked antibody or antigen-binding fragment of any one of claims 1-51.

62. A method of producing the masked antibody or antigen-binding fragment of any one of claims 1-51 comprising culturing the host cell of claim 60 or claim 61.

63. The method of claim 62, further comprising isolating the masked antibody or antigen-binding fragment thereof.

64. A pharmaceutical composition comprising the masked antibody or antigen-binding fragment of any one of claims 1-51 or the immunoconjugate of any one of claims 52-57 and a pharmaceutically acceptable carrier.

65. The masked antibody or antigen-binding fragment of any one of claims 1-51, the immunoconjugate of any one of claims 52-57, or the pharmaceutical composition of claim 64 for use in therapy.

66. The masked antibody or antigen-binding fragment of any one of claims 1-51, the immunoconjugate of any one of claims 52-57, or the pharmaceutical composition of claim 64 for use in treating cancer, an autoimmune disease, or an infection.

67. Use of the masked antibody or antigen-binding fragment of any one of claims 1-51, the immunoconjugate of any one of claims 52-57, or the pharmaceutical composition of claim 64 for the preparation of a medicament for use in therapy.

68. Use of the masked antibody or antigen-binding fragment of any one of claims 1-51, the immunoconjugate of any one of claims 52-57, or the pharmaceutical composition of claim 64 for the preparation of a medicament for use in treating cancer, an autoimmune disease, or an infection.

69. A method comprising administering to a subject in need thereof the masked antibody or antigen-binding fragment of any one of claims 1-51, the immunoconjugate of any one of claims 52-57, or the pharmaceutical composition of claim 64.

70. A method of treating cancer, an autoimmune disease, or an infection comprising administering to a subject in need thereof the masked antibody or antigen-binding fragment of any one of claims 1-51, the immunoconjugate of any one of claims 52-57, or the pharmaceutical composition of claim 64.

71. The method of claim 70, which is a method of treating cancer.

72. The method of claim 71, wherein the cancer is a solid cancer, a soft tissue cancer, or a hematopoietic cancer.

73. The method of claim 71 or claim 72, wherein the cancer is selected from lung cancer, pancreatic cancer, breast cancer, liver cancer, ovarian cancer, testicular cancer, kidney cancer, bladder cancer, spinal cancer, brain cancer, cervical cancer, endometrial cancer, colorectal cancer, anal cancer, endometrial cancer, esophageal cancer, gallbladder cancer, gastrointestinal cancer, gastric cancer, sarcoma, head and neck cancer, melanoma, skin cancer, prostate cancer, pituitary cancer, stomach cancer, uterine cancer, vaginal cancer, thyroid cancer, a sarcoma, soft tissue sarcoma, osteosarcoma, a lymphoma, diffuse large B-cell lymphomas (DLBCL), follicular lymphoma, myelodysplastic syndrome (MDS), Hodgkin's disease, a malignant lymphoma, non-Hodgkin's lymphoma, Burkitt's lymphoma, multiple myeloma, Richter's Syndrome, a leukemia, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMoL).
